(19) United States
(12) Patent Application Publication
(10) Pub. No.: US 2024/0408212 A1
(43) Pub. Date: Dec. 12, 2024

(54) PHARMACEUTICAL COMPOSITION COMPRISING BIOPHARMACEUTICAL DRUG COMPOUNDS

(71) Applicant: OREXO AB, Uppsala (SE)

(72) Inventors: Jonas SÄVMARKER, Uppsala (SE); Robert RÖNN, Uppsala (SE)

(21) Appl. No.: 18/712,027

(22) PCT Filed: Nov. 25, 2022
(86) PCT No.: PCT/GB2022/052985

§ 371 Date: May 21, 2024

(30) Foreign Application Priority Data
Nov. 25, 2021 (GB) 2117005.5
Jun. 1, 2022 (GB) 2208144.2
Sep. 12, 2022 (GB) 2213306.0

(51) Int. Cl. A61K 47/26; A61K 9/00; A61K 9/14; A61K 38/43; A61K 39/395; A61K 47/36

(52) U.S. Cl. CPC A61K 47/26; A61K 9/0043; A61K 9/14; A61K 38/43; A61K 39/395; A61K 47/36

(57) ABSTRACT

According to the invention, there is provided a which composition is in the form of an amorphous, mono-particulate powder comprising a mixture of: (a) a pharmacologically-effective dosage amount of at least one biopharmaceutical drug compound; and (b) a pharmaceutically-acceptable carrier material, which carrier material comprises a combination of a disaccharide and a polymeric material. Preferred pharmaceutically-acceptable carriers in this regard include lactose or trehalose and dextrins (e.g. maltodextrins). Compositions may further comprise one or more alkyl saccharides. Preferred alkyl saccharides include sucrose esters, such as sucrose monolaurate. Powder compositions may be produced by spray-drying the various components together in combination.

This invention relates to new pharmaceutical compositions comprising biological agents that are useful in a variety of medical conditions. The invention also relates to methods of manufacturing such compositions and formulating them into dosage forms.

### PRIOR ART AND BACKGROUND

The listing or discussion of an apparently prior-published document in this specification should not necessarily be taken as an acknowledgement that the document is part of the state of the art or common general knowledge.

Among the various well-known routes of drug delivery, peroral delivery to the gastrointestinal tract is the most common. It is generally regarded as being the most favoured by the patients and practitioners.

However, peroral drug administration is known to have drawbacks, including the fact that active ingredients are necessarily subject to hepatic first-pass metabolism, enzymatic degradation within (and outside) the gastrointestinal tract, and have limited access to the blood brain barrier (BBB) in order to treat diseases of the central nervous system (CNS). These factors may not only affect the efficacy of certain drugs but also, in some cases, disqualify peroral drug delivery as an administration route altogether.

Peroral administration to the gastrointestinal tract has the additional disadvantage that it requires absorption of active ingredients through the intestines as part of the digestive process, which takes time. Additionally, high doses of active ingredients are often needed (due to low bioavailability) which carries the risk of more side effects and/or increased safety issues.

Finally, other limitations of peroral formulations which limits their long-term usage is microbial instability. This may require the use of preservatives to control, which can lead to irritation and give rise to sensitization/allergic effects.

In the treatment of certain conditions, such as acute disorders, a more rapid onset of pharmacological effect than may be provided by peroral drug delivery is often highly desirable.

In such cases, administration principles in which drugs are immediately absorbed into systemic circulation is more likely to lead to a rapid onset of action. Although this can be done via parenteral administration (such as subcutaneous or intravenous injection), such delivery means are inconvenient, and are sometimes very difficult and/or impossible for patients to do, requiring time-consuming intervention by physicians to ensure compliance and to avoid effects that are either unwanted or detrimental.

Transmucosal administration of active ingredients is a viable alternative to parenteral administration. It gives rise to the possibility of delivering drug molecules directly into systemic circulation through mucosal membranes (e.g. rectally, sublingually, buccally, pulmonarily and intranasally), and may lead to advantages, such as increased patient compliance, improved drug bioavailability and therefore lower doses, a more rapid onset of action and reduced side effects.

However, transmucosal administration of drugs presents its own, quite distinct problems. Unlike the gastrointestinal tract, which is a large organ that contains a relatively large amount of biological fluids, spaces such as the oral and nasal cavities are relatively small and contain much lower amounts of bodily fluids, such as saliva and/or mucous. This inevitably provides a considerable limitation on the amount of active ingredient that can be administered in a single dose.

Furthermore, although it is a dynamic system, the gastrointestinal tract is, in the main part, something of a‘closed’ system. Conversely, the rapid clearance mechanisms that take place in both the oral and nasal cavities means that the time that is often available for absorption across a mucosal surface, for an already more limited amount of drug, is also limited.

Numerous formulation principles have been put forward to solve this problem, including, for example, bioadhesive formulation principles, such as buccal patches for oromucosal drug delivery (see, for example, Shojaei, J. Pharm. Pharmaceutical Sci., 15, 19 (1998) and Gandhi, Advanced Drug Delivery Reviews, 43, 67 (1994)), as well as in situ gelling compositions for intranasal drug delivery (see, for example, Bertan et al, Eur. J. Pharm. Sci., 27, 62 (2006)).

Transmucosal drug delivery systems that are in the solid state may present a significant advantage in allowing for higher drug loadings in the formulation. However, although solid drug delivery compositions are far more common when administering to rectal, buccal, sublingual and pulmonary mucosae, it remains the case that the vast majority of intranasal drug delivery systems are presented in the form of liquid sprays, typically aqueous solutions, wherein drug solubility plays yet another limiting factor in the amount of drug that is available for absorption.

That liquid sprays for intranasal delivery are almost ubiquitous is because formulating solid pharmaceutical formulations in form of a nasal powder is not easy. Unlike powders that are frequently employed for inhalation of active ingredients into the lungs, there are very few commercially-available intranasal powder formulations.

When formulated as dry powders, pulmonary drug delivery compositions typically take the form of ‘aggregate’ mixtures that include micronized particles of API on larger carrier particles. These aggregates are intended to dissociate/break up upon inhalation or actuation of a device, depositing only the fine particles of active ingredients in the lung.

However, such drug delivery systems are understood not to work effectively in the case of intranasal drug delivery. This is because the presence of such fine particles leads to a significant risk of lung exposure, which is not the intended site of administration. If drug particle sizes were increased to avoid this problem, it would likely lead to difficulties in ensuring appropriate interactions in the heterogeneous ‘interactive’ mixture, which depends on substantial differences in sizes of the two components to ensure interaction, in turn leading to potential manufacturing issues, such as segregation during filling. Attempting to compensate for this by correspondingly increasing carrier particle size would not necessarily solve the problem, but would necessarily increase the mass of inactive excipients in an already finitely limited total mass of dosage form, potentially resulting in a reduction in the dose of active ingredient.

The difficulties of formulating dry powders for intranasal delivery are dealt with in US Patent Application US 2005/001411 A1. In this document, it is stated that powders for nasal administration need to be fine enough so that they can be efficiently conveyed by a flow of gas and efficiently deposited in the nose, yet also coarse enough to facilitate the introduction of the powder into an appropriate powder device, which is always needed for intranasal administration. US 2005/001411 A1 apparently solves this problem by making loosely formed secondary particles (aggregates) of primary particles comprising active ingredients. The aggregates have dimensions that are a few hundreds of microns, and this is said to enable more efficient loading into an appropriate intranasal administration (an applicator, dispenser or insufflator) device. Upon actuation of such a device, and administration of the composition, the aggregates apparently quickly break up into the primary particles of active ingredients. These primary particles are of a size that is just a few microns, which is stated to facilitate their dissolution and, thereafter, intranasal absorption of active ingredient.

As stated above, transmucosal (e.g. intranasal) delivery of drugs intended for systemic absorption avoids the first pass metabolism that is inevitably a component of peroral administration. Drug metabolism occurs through chemical reactions with enzymes that are capable of altering an active ingredient's chemical structure, physical structure and/or biological activity.

Because most drugs are organic molecules that contain functional groups that are capable of undergoing such chemical reactions, they are often susceptible to some form of chemical decomposition when they come into contact with substances that are capable of interacting with those functional groups outside of the body.

Such chemical transformation is typically classified as chemical ‘degradation’ in the pharmaceutical field, because it can often lead to a loss of efficacy or, in extreme situations, toxic by-products, either or both of which may lead to a drug being ineffective and/or harmful to patients.

How rapidly such degradation can occur depends upon how inherently chemically-unstable the drug compound is in the first place, the way that it is formulated and the conditions of its storage. Often high temperatures and humidities can lead to accelerated degradation.

Such a loss of chemical integrity is measurable, and is why all pharmaceutical products have shelf-lives printed on their label and/or embossed on their packaging. It is also why certain prescribed medicines contain specific printed information in packaging inserts regarding appropriate storage conditions.

As a rule of thumb, the more complex the active ingredient, the more the likelihood of chemical or physical integrity leading to biological inactivation being a problem. In this respect, biologic active drugs/active pharmaceutical ingredients (APIs), such as vaccines, enzymes, antibodies/parts thereof, and antibody analogues and mimetics present particular issues, for example because of their higher order (e.g. tertiary) structures, and the interplay between being in a proper conformation and effective function.

Furthermore, despite being proven to be increasingly useful in the treatment of a broad range of diseases and disorders, compared to other APIs that are small molecules, certain biological agents or biologics, such as antibodies/parts thereof, may not be especially potent and may require high doses to achieve a notable biological effect.

As is summarised by Kou and Zhou in Chapter 16 of the textbook Amorphous Solid Dispersions, Shah et al (Eds.), Springer (2014), if a drug is formulated in an amorphous, as opposed to a crystalline, physical state, it is typically presented in a higher energy state, and is thus likely to be more chemically and physically unstable, presenting challenges to pharmaceutical formulators.

Chemical stability is thus often improved by presenting a drug (particularly one that is a small molecule) in a crystalline state, often through salt formation. However, it is not a straightforward matter, and is indeed usually not an option, to present a large (macromolecular) biologic in the form of such a salt.

Thus, in view of all of the aforementioned potential advantages that it offers, there remains a need for improved solid (e.g. powder-based) transmucosal and especially intranasal drug delivery systems.

In particular, there remains a significant unmet clinical need in the field of drug delivery, for a powdered drug delivery composition that:
(i) is both physically and chemically stable; and
(ii) provides active ingredient:
   at a sufficient dose; and/or
   if intended for systemic administration, in a form in which it is permeable enough
to provide a required therapeutic effect (such as speed of onset and/or access to a drug target) at the (relatively speaking) low doses that are possible, and short residence times that are available, in the transmucosal context, such as within the nasal cavity.

In addition to the above, in the more specific field of intranasal drug delivery, there remains a significant unmet clinical need for such a drug delivery composition that comprises particles of an appropriate size to enable both the efficient:
filling of a drug delivery device; and
deposition within the relevant (e.g. nasal) cavity.

Intranasal dry powder formulations are known from inter alia international patent applications WO 2010/142696 and WO 2019/038756, U.S. Pat. No. 10,653,690 B1 and US patent application US 2018/0092839A.

Spray-drying of biologics has been disclosed in, for example, Bürki et al, Int. J. Pharm., 408, 248 (2011) and Lipiäinen et al, ibid., 543, 21 (2018)).

We have now found that it is possible to formulate certain biologic active ingredients (i.e. biological agents or biologics) in the form of amorphous dry powder compositions by way of a process that, for example, spray-dries those active ingredients along with a specific combination of carrier materials, as disclosed hereinafter. Such compositions may provide for surprising and substantial improvements in stability of those active ingredients before administration, without significant loss in pharmacological and/or biological activity of relevant active ingredients. Such compositions may in addition provide for improved bioavailability and/or speed of absorption of those active ingredients following administration.

### DISCLOSURE OF THE INVENTION

According to a first aspect of the invention, there is provided a pharmaceutically-acceptable composition in the form of a solid, amorphous, mono-particulate powder comprising a mixture of:
(a) a pharmacologically-effective dosage amount of at least one biopharmaceutical drug compound; and
(b) a pharmaceutically-acceptable carrier material, which carrier material comprises a combination of a disaccharide and a polymeric material, which pharmaceutically-acceptable compositions are referred to hereinafter together as ‘the compositions of the invention’.

Compositions of the invention are in the form of an amorphous, mono-particulate powder. By ‘mono-particulate’, we mean that the plurality of particles that form the powdered compositions of the invention comprise a homogeneous or a heterogeneous mixture, in which biopharmaceutical drug compound(s) (also referred to herein as ‘active ingredient(s)’, ‘pharmaceutically-active ingredient(s), ‘pharmacologically-active compound(s)’ and/or ‘drug(s)’) are encapsulated in an amorphous state within the carrier material as defined above, optionally in the presence of other ingredients. The particles of the powdered compositions of the invention are thus presented as an amorphous composite of active ingredient, the carrier material and, optionally, other ingredients.

By being amorphous in their nature, compositions of the invention may be wholly amorphous and/or may be predominantly amorphous (for example more than about 50% by weight, such as more than about 75% by weight, including more than about 80% by weight, such as more than about 90% by weight, or 95% by weight, including more than about 99% by weight amorphous). In the alternative, compositions of the invention may be less than about 50%, such as less than about 25%, more preferably less than about 20%, for example less than about 10%, including less than about 5%, or less than about 1% crystalline. The degree (%) of crystallinity may be determined by the skilled person using powder X-ray diffraction (PXRD). Other techniques, such as solid-state NMR, FT-IR, Raman spectroscopy, differential scanning calorimetry (DSC) microcalorimetry, and calculations of true density, may also be used.

As described hereinafter, despite being in an amorphous physical state, compositions of the invention exhibit remarkable and unexpected physical and chemical stability, and may thus be provided in the form of pharmaceutical products that show excellent shelf-life when stored under normal storage conditions.

Compositions of the invention are produced at least initially in multiparticulate form (i.e. as powders) by an appropriate technique. In general, appropriate techniques fall into ‘solvent-based’ methods, which include spray-drying, fluidized bed techniques, co-precipitation, supercritical fluid techniques, spray granulation, cryogenic techniques (including freeze-drying), electrospinning and rotating jet techniques, or‘fusion-based’ methods, which include melt granulation, melt extrusion, high-shear mixing (e.g. KinetiSol®), milling and molten material on carrier techniques (e.g. Meltdose®). Preferred methods include freeze-drying and, more preferably, compositions of the invention are made by a process of spray drying.

Such powders may be suitable for delivery via any pharmaceutically-acceptable administration route directly to patients, or may be presented as an intermediate composition that may subsequently be formulated into a pharmaceutically-acceptable dosage form which is to be administered to one or more patients.

In this respect, there is provided a pharmaceutical formulation and/or a pharmaceutically-acceptable dosage form which formulation and/or dosage form is to be administered to a patient, and comprises one or more compositions of the invention. Suitable pharmaceutical dosage forms may thus comprise liquid formulations, such as solutions, which may be prepared by dissolving a composition of the invention (e.g. just prior to administration) in a pharmaceutically-acceptable solvent (such as water), for delivery to such patients for example by injection or by infusion. Such administration means may be useful when the active ingredient is an antibody or the like.

Alternative pharmaceutical dosage forms may comprise liquid or semi-solid formulations, such as liquid suspensions and/or gel compositions which may comprise (e.g. particles of) a composition of the invention that is/are suspended or dissolved in an appropriate liquid or semi-solid carrier which may be loaded into an appropriate dosage form or delivered by, for example, injection or infusion, or may be formed after injection (e.g. subcutaneously or intramuscularly) to form an implant or a depot formulation.

Compositions of the invention may in the alternative be presented as part of an essentially solid pharmaceutical dosage form. The term ‘solid’ will be well understood by those skilled in the art to include any form of matter that retains its shape and density when not confined, and/or in which molecules are generally compressed as tightly as the repulsive forces among them will allow. An essentially solid formulation is thus one that is at least about 80%, such as at least about 90%, including at least about 95% (or at least about 99%) in such a form.

In this respect, compositions of the invention may be provided in any multi-particulate form (e.g. as simple powders, granules, pellets and/or beads), comprising a plurality of particles that may individually and/or may collectively consist essentially of, and/or comprise, one or more compositions of the invention.

Compositions of the invention may thus be presented following their preparation (e.g. by spray-drying) in the form of simple powder mixtures, powder microspheres, coated powder microspheres, a lyophilised liposomal dispersion, or a combination thereof.

If a pharmaceutically-acceptable dosage form of the invention ‘consists essentially of’ the particles of one or more compositions of the invention, this will be understood to mean that that dosage form comprises only one or more compositions of the invention, along with other features and/or components that do not materially affect the basic and novel characteristic(s) of the dosage form. Alternatively, in situations where the dosage forms of the invention ‘consist essentially of’ one or more compositions of the invention, this may be understood to mean that that dosage form comprises at least about 90%, such as at least about 95%, including at least about 97% (e.g. about 99%) by weight of those one or more compositions of the invention in total.

Pharmaceutical dosage forms may in the alternative comprise one or more compositions of the invention, which may be provided in the form of a single unit dosage form, such as a pessary, a suppository or another form of insert, a pill, a capsule, a cake, a patch (e.g. a buccal patch), a film (e.g. an intraoral film) or a tablet (e.g. a sublingual tablet).

Capsules may be prepared by loading a composition of the invention as a spray-dried powder directly into a pharmaceutically-acceptable capsule made from an appropriate material designed for either sublingual or, preferably, peroral delivery, or by mixing a composition along with excipients prior to loading into such a capsule, which may involve a granulation step as described hereinafter, prior to loading into a capsule for such delivery. Peroral delivery may be employed when the active ingredient is, for example, an enzyme.

Compositions of the invention may in this respect be granulated into a pellet or a pill, but they may also be formulated (that is, provided for administration) in the form of a dry, free-flowing powder. By ‘dry’ we include essentially free of water and other liquid solvents, which includes that there is less than about 10%, such as less than about 6%, including less than about 5%, or less than about 4%, more preferably less than about 3%, such as less than about 2%, e.g. less than about 1% of the formulation is a liquid, such as water.

Flowability of powder compositions of the invention may be measured by standard techniques known to those skilled in the art including bulk density measurements, or measurements taken on a powder flow analyser (for example those sold by Stable Micro Systems or Meritics, both UK), including powder flow speed dependence tests, caking tests, cohesion tests, etc. A preferred measurement of flowability is the standard angle of repose, which may be carried out using a revolving cylinder, a fixed funnel or a tilting box.

In the context of the present invention, the term ‘free-flowing’ is intended to include a powder that allows for efficient filling of a composition of the invention into a drug delivery device during manufacturing, and/or provides a sufficient shot weight when expelled from the device (vide infra).

The term may also include that the powder exhibits an angle of repose of no more than about 50°, such as no more than about 45°, including no more than about 40°, for example no more than about 35°, and more particularly no more than about 30°; a bulk density of no less than about 0.3 g/mL, for example no less than about 0.4 g/mL, such as no less than about 0.5 g/mL, and more particularly no less than about 0.6 g/mL; and/or a tap density of no less than about 0.5 g/mL, such as no less than about 0.6 g/mL, for example no less than about 0.7 g/mL, and in particular no less than about 0.8 g/mL.

Appropriate techniques for making dosage forms comprising dry powders or granulates include simple dry mixing, granulation (including dry granulation, wet granulation, melt granulation, thermoplastic pelletising, spray granulation), extrusion/spheronisation or, more preferably, freeze-drying or spray-drying (vide infra).

Dry granulation techniques are also well known to those skilled in the art and include any technique in which primary powder particles are aggregated under high pressure, including slugging and roller compaction, for example as described hereinafter.

Wet granulation techniques are well known to those skilled in the art and include any technique involving the massing of a mix of dry primary powder particles using a granulating fluid, which fluid comprises a volatile, inert solvent, such as water, ethanol or isopropanol, either alone or in combination, and optionally in the presence of a binder or binding agent. The technique may involve forcing a wet mass through a sieve to produce wet granules which are then dried, preferably to a loss on drying of less than about 3% by weight.

Melt granulation will be known by those skilled in the art to include any technique in which granules are obtained through the addition of a molten binder, or a solid binder which melts during the process (which binder materials may comprise the pharmaceutically acceptable carrier materials of the composition of the invention). After granulation, the binder solidifies at room temperature. Thermoplastic pelletising will be known to be similar to melt granulation, but in which plastic properties of the binder are employed. In both processes, the agglomerates (granules) obtained comprise a matrix structure.

Extrusion/spheronisation will be well known to those skilled in the art to include any process involving the dry mixing of ingredients, wet massing along with a binder, extruding, spheronising the extrudate into spheroids of uniform size, and drying.

Spray granulation will be known by those skilled in the art to include any technique involving the drying of liquids (solutions, suspensions, melts) while simultaneously building up granulates in a fluid bed. The term thus includes processes in which foreign seeds (germs) are provided upon which granules are built up, as well as those in which inherent seeds (germs) form in the fluid bed due to abrasion and/or fracture, in addition to any spray coating granulation technique generally. The sprayed liquid coats the germs and assists further agglomeration of particles. It is then dried to form granules in the form of a matrix.

The term ‘freeze drying’ includes lyophilisation or cryodesiccation, and any low temperature desolvatization (e.g. dehydration) process, in which product is frozen, pressure is lowered, and the frozen solvent (e.g. water) is removed by sublimation.

Compositions of the invention may in the alternative be provided in the form of a tablet for peroral, buccal and/or sublingual use. Such tablets may be formed for example by direct compression/compaction of a composition of the invention, optionally following mixing it together with one or more appropriate excipients, such as a diluent, a disintegrant, a glidant and/or a lubricant, and may be achieved using techniques such as those described in, for example, Pharmaceutical Dosage Forms: Tablets. Volume 1, 3^{rd} Edition, Augsburger et al (eds.), CRC Press (2008) and the documents cited therein. Suitable compacting equipment includes standard tabletting machines, such as the Kilian SP300 or the Korsch EK0, XP1, XL 100, and XL 200.

Suitable disintegrants (as defined in, for example, Rowe et al, Handbook of Pharmaceutical Excipients, 6^{th} ed. (2009)) that may be employed in tablets include cellulose derivatives such as hydroxypropyl cellulose (HPC), low substituted HPC, methyl cellulose, ethyl hydroxyethyl cellulose, carboxymethyl cellulose calcium, carboxymethyl cellulose sodium, microcrystalline cellulose, modified cellulose gum; starch derivatives such as moderately cross-linked starch, modified starch, hydroxylpropyl starch and pregelatinized starch; and other disintegrants such as calcium alginate, sodium alginate, alginic acid, chitosan, colloidal silicon dioxide, docusate sodium, guar gum, magnesium aluminum silicate, polacrilin potassium and polyvinylpyrrolidone. Combinations of two or more disintegrants may be used. Preferred disintegrants include so-called ‘superdisintergrants’ (as defined in, for example, Mohanachandran et al, International Journal of Pharmaceutical Sciences Review and Research, 6, 105 (2011)), such as cross-linked polyvinylpyrrolidone, sodium starch glycolate and croscarmellose sodium. Combinations of two or more superdisintegrants may be used.

When disintegrants and/or superdisintegrants are employed in tablets, they may be employed in an (e.g. total) amount of between 0.5 and 15% by weight based upon the total weight of a composition. A preferred range is from 1 to 8%, such as from about 2 to about 7% (e.g. about 5%, such as about 4%) by weight.

If present, binder is preferably employed in an amount of between 0.5 and 20% by weight based upon the total weight of the tablet formulation. A preferred range is from 1.0 to 15%, such as from about 2.0 to about 12% (e.g. about 10%) by weight. Suitable binders include cellulose gum and microcrystalline cellulose.

As described herein, compositions of the invention are preferably made by a process of spray-drying.

Whether in the form of a powder or otherwise, dosage forms comprising compositions of the invention may otherwise be prepared by standard techniques, and using standard equipment, known to the skilled person. In this respect, the compositions of the invention may be combined with conventional pharmaceutical additives and/or excipients used in the art for relevant preparations, and incorporated into various kinds of pharmaceutical preparations using standard techniques in order to make dosage forms comprising compositions of the invention (see, for example, Lachman et al, ‘The Theory and Practice of Industrial Pharmacy’, CBS, 4^{th} edition (2015); ‘Remington: The Science and Practice of Pharmacy’, Troy (ed.), Elsevier, 23^{rd} edition (2020); and/or ‘Aulton's Pharmaceutics: The Design and Manufacture of Medicines’, Taylor and Aulton (eds.), Elsevier, 5^{th} edition, 2017).

However they are manufactured, it is preferred that compositions of the invention are suitable for, and/or are formulated for, transmucosal delivery of the active ingredient into systemic circulation (and in the case of e.g. vaccines, generate a systemic immune response), or, again in the case of e.g. vaccines, to generate an immune response in the local microenvironment.

The term ‘transmucosal’ will be understood by those skilled in the art to mean that, however it is administered to a patient, a composition is presented at a relevant mucosal surface in such a form that the active ingredient(s) may be absorbed across that mucosal surface following its dissolution. Relevant mucosal surfaces thus include the oral, nasal, ocular, vaginal, cervical, pulmonary and/or anorectal mucosae, more particularly the oral mucosa (including buccal and sublingual mucosae) and the nasal mucosa.

Thus, dosage forms comprising a composition of the invention may be directly administered to a mucosal surface (including pulmonarily, rectally, vaginally, buccally, sublingually or intranasally) of a patient for transmucosal delivery of active ingredients. Pulmonary and, in particular, intranasal administration is particularly useful when the active ingredient is a vaccine, in which latter case transmucosal absorption may not be necessary to provoke an immune response to the antigen.

If administered to the sublingual mucosa, compositions of the invention may be in the form of e.g. sublingual tablets as described above, which may comprise disintegrants or disintegrating agents (which may be defined as any material that is capable of accelerating to a measurable degree the disintegration/dispersion of such composition of the invention), which may be achieved, for example, by the material being capable of swelling and/or expanding when placed in contact with aqueous media, as described hereinafter.

Alternatively, compositions of the invention may be administered sublingually in the form of a powder as described herein, which may be emptied into the mouth and under the tongue from an appropriate receptacle, such as a capsule or a sachet.

If compositions of the invention are suitable for, and/or are formulated for sublingual or, more notably, intranasal administration, then they are preferably administered in the form of a powder composition in which the dosage amount of the active ingredient(s) is no more than about 100 mg or no more than 10⁹ international unit (IU). Such sublingual and/or nasal powder compositions may comprise a composition of the invention admixed with other excipients, or may consist essentially of a composition of the invention as hereinbefore defined.

Compositions of the invention that are suitable for, and/or are formulated for, intranasal administration are preferably provided by way of a dosing means that is suitable for nasal delivery. Such a dosing means may contain one spray-dried powder composition of the invention, or may contain two or more such compositions. In the latter instance, the dosing means contains two or more dosing amounts of said composition of the invention, which dosing amounts will each contain a pharmacologically-effective dose of the active ingredient(s).

Two or more compositions of the invention may be administered intranasally, either by repeated actuation of a device that either comprises, or is in communication with, that dosing means. Compositions of the invention may therefore be presented within an appropriate device (e.g. a nasal applicator or dispenser (insufflator), for example as described hereinafter), and/or may be presented within a container or a reservoir that is part of, is adjunct to, and/or is suitable for being placed adjunct to, such an applicator. Such a container or reservoir may contain the one or more compositions of the invention, each containing a pharmacologically-effective dosage amount of said active ingredient.

In this way, appropriate dosing means and/or nasal applicators may be actuated only once to deliver a single composition of the invention comprising an appropriate dose of an active ingredient following that actuation (i.e. a single-use dosing unit), may be actuated more than once to deliver two or more compositions of the invention, each comprising an appropriate dose of active ingredient, upon each such actuation (i.e. a multiple-use dosing unit), and/or applicators may be re-filled with a replacement source of composition(s) of the invention (e.g. a container or reservoir), comprising one or more such compositions, to provide for single and/or multiple doses and/or dosing regimens.

Compositions of the invention may thus be administered in the form of a plurality of particles, which particles may individually and/or collectively consist of, and/or comprise, compositions of the invention.

Compositions of the invention are thus prepared (initially) in the form of solid, dry, free-flowing, multi-particulate powders, as described hereinbefore.

As stated above, compositions of the invention are provided in the form of amorphous, mono-particulate powders. They are not composed of physical associations of two or more discrete, separate sets of particles of different ingredients in the form of a mixture, such as an ordered, or interactive, mixture of smaller particles of active ingredient(s) associated with larger, but separate and chemically distinct, particles of carrier substances. That said, compositions of the invention may be provided as small particles which may subsequently be adhered to separate, larger carrier particles in an interactive mixture, and such a presentation may be useful if the dosage form that is intended for inhalation, for example to the lung, (see e.g. J. Drug Delivery, Art. ID 5635010, 1-19 (2018)).

As mentioned hereinbefore, the process of making compositions of the invention enables the formation of pharmaceutical products that show excellent shelf-life, in terms of both physical and chemical stability, when stored under normal storage conditions, as defined herein.

Compositions of the invention are preferably prepared by a process of spray-drying. The process of ‘spray-drying’ will be understood by the skilled person to include any method of producing a dry powder from a liquid, including a solution or a suspension (including a slurry) that involves rapid drying using hot gas to convert a stream of liquid into vaporized solvent and particles of solid, which solid particles comprise the solute that was previously dissolved in a solution, and/or particles that were previously suspended in the evaporated liquid.

Appropriate spray-drying equipment includes some form of atomization means, such as a spray nozzle, which disperses the liquid into a spray with a relatively uniform droplet size. Such means may include any means that is capable of producing a dry, free-flowing powder, and may include high pressure swirl nozzles, rotary disks and/or atomizer wheels, high pressure single fluid nozzles, two-fluid nozzles and/or ultrasonic nozzles.

The spray-dryer may be a single effect or a multiple effect spray-dryer, and may comprise an integrated and/or an external vibrating fluidized bed, a particle separator, and/or a collection means which may be a drum or a cyclone.

According to a further aspect of the invention, there is provided a process for the manufacturing of a composition of the invention, wherein said process comprises the steps of:
i) mixing together the one or more active ingredients and pharmaceutically-acceptable carrier material, in an appropriate volatile solvent,
ii) spray-drying the mixture from step i).

Preferred volatile solvents include water, or organic solvents, such as lower alkyl alcohols (e.g. methanol, isopropanol or, more especially, ethanol), hydrocarbons (e.g. C₅₋₁₀ alkanes), haloalkanes (e.g. dichloromethane), dimethylformamide, dimethylsulfoxide, ethyl acetate, acetone, etc., or mixtures thereof.

We prefer that mixing together the one or more active ingredients, pharmaceutically-acceptable carrier material(s) as defined herein, and other optional ingredients as described herein (for example alkyl saccharides as described hereinafter), with the solvent results in a solution that can be spray-dried.

Appropriate pharmaceutically-acceptable carrier materials that may be employed in compositions of the invention include relevant materials that, in the appropriate combination, are suitable (and/or approved) for pharmaceutical use and/or for transmucosal (e.g. sublingual or, notably, intranasal) delivery, and are capable of maintaining their physical and/or chemical integrity, and/or do not affect the physical and/or chemical integrity of any active ingredients and/or any other ingredients that are or may be present in the composition (such as alkyl saccharide), in the solid state, under normal storage conditions.

It is well known that significant difficulties may be experienced in attempting to obtain both chemically- and physically-stable solid compositions, such as powders. If the physical form of a composition changes under normal storage conditions (e.g. from a free-flowing powder to an agglomerated mass that is difficult to discharge), it will likely lead to non-reproducibility of dose of active ingredient. This is particularly so when dispensing a composition from, or via, a nasal applicator as described herein, where such agglomeration may result in the complete inability to dispense the active ingredient.

Compositions of the invention may this have a minimum shot weight, as measured by individual powder shot weight relative to target weight of about 80%, such as about 85% (e.g. about 90%) up to about 120% (e.g. about 115%, such as about 110%), and/or a mean powder shot weight relative to target weight of about 85%, such as about 90% (e.g. about 95%) up to about 115% (e.g. about 110%, such as about 105%).

Similarly, for multiple dose units containing two or more doses of a composition, such stability is critical to ensure reproducibility of the dose of active ingredient over time. Either of these problems may have a detrimental effect on a subject's health, and/or put a subject's well-being at significant risk.

For certain compositions of the invention, exposure to atmospheric water may result in powder compositions that are less solid-state stable. For example, exposure to certain (e.g. higher) relative humidities may affect the physical form of the composition, for example by deliquescence, and/or by lowering glass transition temperatures of compositions, and/or individual components of the compositions, such as carrier materials, or in another way.

Accordingly, compositions of the invention, and pharmaceutical formulations and dosing means (such as nasal applicators) including them, are preferably packaged within containers that substantially prevent the ingress of atmospheric water under the storage conditions defined herein. Such containers may include packaging materials, such as blister packs for tablets and capsules and heat-sealed aluminium pouches and/or thermoformed plastics. Such containers may also comprise a desiccant, such as silica gel and/or appropriate molecular sieves, with a pore size of e.g. 3 Å or 4 Å.

The phrase ‘maintaining physical and chemical integrity’ essentially means chemical stability and solid-state stability.

By ‘chemical stability’, we include that any composition of the invention may be stored in isolated solid form, when formulated into a pharmaceutical formulation or dosage form, and/or when loaded into a pharmaceutical dosing means, such as a nasal applicator or a reservoir therefor (with or without appropriate pharmaceutical packaging) or otherwise, under normal storage conditions, with an insignificant degree of chemical degradation or decomposition of either the composition per se or the active ingredient included therein.

The term ‘chemical stability’ may also include ‘stereochemical’ and/or ‘configurational’ stability, by which we mean resistance to stereochemical conversion, such as racemisation, at one or more chiral centres within a biopharmaceutical drug compound’ molecule.

By ‘physical stability’ or ‘solid-state stability’, we include that any composition of the invention may be stored in an isolated solid form, when formulated into a pharmaceutical formulation or dosage form, and/or when loaded into a pharmaceutical dosing means, such as a nasal applicator or a reservoir therefor (with or without appropriate pharmaceutical packaging) or otherwise, under normal storage conditions, with an insignificant degree of solid-state transformation (e.g. crystallisation, recrystallisation, loss of crystallinity, solid-state phase transition (e.g. between a glassy or a rubbery state, or to an agglomerated form)), hydration, dehydration, solvatisation or desolvatisation of either the composition per se or the active ingredient included therein. By ‘physical stability’, we also include retaining the correct conformation of a folded protein, and/or the retention of biological activity as defined hereinafter.

Examples of ‘normal storage conditions’ for compositions of the invention, whether in the form of a pharmaceutical formulation or dosage form, and/or when loaded into a pharmaceutical dosing means loaded into applicators, devices, drug reservoirs (such as canisters or containers) or otherwise, include temperatures of between about −50° C. and about +80° C. (preferably between about −85° C. (such as −25° C.) and about +75° C., such as about 50° C.), and/or pressures of between about 0.1 and about 2 bars (preferably atmospheric pressure), and/or exposure to about 460 lux of UV/visible light, and/or relative humidities of between about 5 and about 95% (preferably about 10 to about 40%), for prolonged periods (i.e. greater than or equal to about twelve, such as about six months).

Under such conditions, compositions of the invention (and/or active ingredients contained therein) whether included in a pharmaceutical dosing means, such as a nasal applicator or a reservoir therefor (with or without appropriate pharmaceutical packaging) or otherwise, may be found to be less than about 15%, more preferably less than about 10%, and especially less than about 5%, chemically degraded/decomposed, and/or solid-state transformed, as appropriate. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Notwithstanding the above definition of ‘normal storage conditions’, compositions of the invention (and/or active ingredients contained therein) may be less than about 5%, such as less than about 4% (including less than about 3%, such as less than about 2.5% (e.g. about 2%), including less than about 1.5% and even less than about 1%) chemically (and/or stereochemically) degraded after storage for:
(a) at least about 3 months, including at least about 6 months or at least about 12 months, at about −85° C., including −25° C., such as 0° C. or room temperature (e.g. about 20° C. or about 25° C.) or about 40° C. and 75% relative humidity;
(b) at least about 18 months, such as at least about 24 months, including at least about 36 months at below about −85° C., including −25° C., such as 0° C. or room temperature (e.g. about 20° C. or about 25° C.) or about 30° C. (at e.g. a relative humidity of about 65%, such as about 60%); and/or
(c) at least about 18 hours at above about 1 million lux of UV light.

Such chemical and, particularly, physical stability is of importance in a solid-state composition, such as a powder, to ensure that the appropriate dose is delivered to the patient.

Compositions of the invention can therefore be stored within a dosage form, such as an applicator or a reservoir therefor (with or without appropriate pharmaceutical packaging) or otherwise, at any temperature (e.g. as low as about −85° C., such as −25° C., including 0° C. or 20° C.) up to about 25° C. (e.g. up to about 30° C.), preferably with excursions up to about 40° C. or even up to about 50° C.

Particularly preferred pharmaceutically-acceptable carrier materials that may be employed to produce compositions of the invention, and which possess the desirable characteristics mentioned herein, include, for the disaccharide component, maltitol, sucralose, sucrose, isomalt, maltose, lactose and, particularly, trehalose.

For the polymeric material component, preferred pharmaceutically-acceptable carrier materials that may be employed to produce compositions of the invention, and which possess the desirable characteristics mentioned herein, include cellulose and its derivatives, such as sodium carboxymethyl cellulose, ethyl cellulose, cellulose acetate, hydroxypropylmethyl cellulose (hypromellose, HPMC), hydroxyethyl cellulose (HEC), hydroxypropyl cellulose (HPC), methyl cellulose (MC), ethyl hydroxyethyl cellulose, carboxymethyl cellulose (CMC), modified cellulose gum, microcrystalline cellulose and sodium carboxymethyl cellulose; starches, such as rice starch, tapioca starch, wheat starch and, more particularly, corn starch and potato starch; starch derivatives, such as pregelatinized starch, carboxymethyl starch, as well as moderately cross-linked starch, modified starch and sodium starch glycolate; polysaccharides, including dextran, pullulan, inulin and dextrins, such as dextrin, cyclodextrins and linear or branched dextrins, such as maltodextrins; powdered tragacanth; waxy excipients, such as cocoa butter and suppository waxes; polyols, such as solid polyethylene glycols; acrylic polymers, such as carbomer and its derivatives; polyvinylpyrrolidone (povidone, PVP); crosslinked polyvinylpyrrolidone; polyethylene oxide (PEO); chitosan (poly-(D-glucosamine)); natural polymers, such as gelatin, sodium alginate, pectin; scleroglucan; xanthan gum; guar gum; poly co-(methylvinyl ether/maleic anhydride); and croscarmellose (e.g. croscarmellose sodium). Hypromellose acetate succinate (HPMCAS), copovidone and polyvinyl alcohol (PVA, or PVOH) may also be mentioned. More preferred polymeric materials include sodium carboxymethyl cellulose, sodium starch glycolate, polyvinylpyrrolidone and, particularly, hydroxypropylmethyl cellulose (such as hypromellose 2906, preferably hypromellose 2910 (i.e. ‘E’-types), and more preferably USP/NF hypromellose 2208 (i.e. ‘K’-types)), and the like, or, particularly, polysaccharides, such as dextrins, including cyclodextrins (e.g. α-, β- and γ-cyclodextrins and derivatives thereof, such as, 2-hydroxypropyl-γ-cyclodextrin, sulfobutylether β-cyclodextrin sodium salt, randomly methylated β-cyclodextrin, branched β-cyclodextrin and the like and, particularly, 2-hydroxypropyl-β-cyclodextrin); and linear or branched dextrins, such as maltodextrins.

In any event, suitable polymers for use in compositions of the invention should have a molecular weight that is high enough such that, when it is employed in any given amount in combination with a disaccharide, it is capable of forming a suitable carrier material for the active ingredient.

For any given polymer, polymer chain length (and therefore molecular weight) is directly proportional to its viscosity. Put another way, the viscosity of a solution of that polymer is proportional to the molecular weight or chain length of the specific polymer.

In this respect, it may be preferred that the polymer has a relative viscosity value at 20° C. of no more than about 1000 (more preferably no more than about 120, such as no more than about 60, and particularly no more than about 10) mPa*s, as measured, for any given and essentially:
(a) water-soluble polymer, as a 2 wt % solution of the polymer in water by the standard USP methods for viscosity, i.e. <911> Method I, and/or <912> Method I; and
(b) water-insoluble polymer, as a 5 wt % solution of the polymer in a suitable organic solvent, such as acetone, methanol, ethanol, isopropyl alcohol, ethyl acetate, acetonitrile, dichloromethane, toluene and mixtures thereof, which solvent system may be dry or partly aqueous, by the USP method <911> Method I.

The skilled person will understand which test is more suitable for the polymer tested.

It is preferred that the carrier material is capable of giving rise to a composition of the invention that possesses a glass transition temperature (Tg) that:
(a) enables its production as a hard and/or brittle, ‘glassy’, amorphous, powdered physical form, that can be easily formulated into a pharmaceutical formulation or dosage form, and/or loaded into a suitable dosing means, such as a nasal applicator, or a drug reservoir and/or container within, or adjunct to, such an applicator, as described herein; and
(b) is high enough that, after such a pharmaceutical formulation, dosage form or dosing means, such as an applicator or reservoir, is packaged as described herein, and thereafter subjected to a high external temperature (e.g. up to between about 50° C. and about 80° C.), it remains in that glassy state, rather than being transformed into a more viscous or rubbery state, and/or a crystalline state.

Such extreme external temperatures are often experienced inside vehicles in warm and/or sunny climates, which vehicles will frequently be parked for extended periods of time in full sun, where the resultant heat gain can be enormous. If the Tg of an (e.g. powder) composition of the invention is low, the composition may transform after exposure to such high temperatures to such a viscous/rubbery state, this will give rise to inefficient dosing of the composition, for example inefficient discharging of the composition from a dosing means, such as an applicator or reservoir therefor (and so too the dose(s) of active ingredient) once the dosing means or applicator is actuated. Furthermore, a too low Tg may affect the disintegration and/or dissolution of compositions of the invention in the form of tablets for sublingual or peroral use.

In this respect, we prefer that the lowest measurable Tg of a composition of the invention is at least about 10° C., such as at least about 15° C., such as at least about 20° C., including at least about 25° C., such as at least about 35° C., including at least about 40° C., such as at least about 50° C., such as at least about 55° C., including at least about 60° C., when measured at a relative humidity of up to about 35%, such as up to about 30%, including up to about 25% (e.g. up to about 20%, such as less than about 15%, e.g. less than about 10%). By ‘lowest measurable Tg’, we include that the composition of the invention may comprise particles that are heterogenous in their nature. In particular, particles may comprise discrete regions of the carrier materials, or composite mixtures thereof, and so may possess individual and separate Tg values. It will be clear to the skilled person that the value of the lowest measurable Tg has a strong impact on the physical stability of the composition.

We have in particular found that compositions of the invention comprising a combination of a disaccharide and a polymer (e.g. HPMC as defined herein) and/or, particularly, a dextrin are capable of giving rise to an appropriate level of physical and chemical stability of compositions and active ingredients when compared to other carrier materials, when employed alone or in isolation.

A particularly preferred combination of carrier materials thus includes a lactose, such as α-D-lactose monohydrate, or, more preferably trehalose, and a dextrin, and especially a cyclodextrin, such as 2-hydroxypropyl-β-cyclodextrin, or a maltodextrin, with a DE above 11, such as maltodextrin 12DE, or above 15, such as maltodextrin 19DE. We have found that such a combination of carrier materials can be spray-dried together along with an active ingredient and also, if present, an alkyl saccharide in appropriate proportions to produce a composition of the invention that possesses both the desired physical and chemical stability under normal storage conditions, as defined herein.

We have found that relative amounts of the disaccharide and the polymer ingredients in the carrier material (and particularly so when the polymer is a dextrin) can be tailored to ensure the required level of physical and/or chemical stability of active ingredient whilst, at the same time, not lowering the Tg of the composition of the invention in such a manner that it affects its physical stability.

We have found that a ratio of between about 50:1 to about 1:50 of disaccharide:polymer (e.g. dextrin) by weight, based on the total weight of the composition, may work depending on the active ingredient that is employed. Preferred ratios are in the range of about 10:1 to about 1:40 (including up to about 1:30 or up to about 1:20), for example between about 7:1, including about 5:1, such as about 4:1, about 3:1 or about 2:1, and about 1:10, such as about 1:8, including about 1:5, for example 1:3 or 1:2, more preferably about 8:1 (e.g. about 7:1, about 3:1, about 2:1 or about 1:1) to about 1:8 (e.g. about 1:3 or about 1:2) of disaccharide:polymer (e.g. dextrin) by weight, based on the total weight of the composition.

A particularly preferred combination of carrier materials thus includes trehalose or a lactose, such as α-D-lactose monohydrate, and a dextrin, and especially a cyclodextrin, such as 2-hydroxypropyl-β-cyclodextrin, or, more preferably, a maltodextrin.

Maltodextrins are classified by DE (dextrose equivalent), with the higher the DE value, the shorter the average length of the glucose chains.

Preferred maltodextrins include those with a DE of between 6 and 15, such as 8 and 12, or above 15, for example up to 47, such as 38, 39, preferably 23, 24, 25 or 26, or, more preferably, 16, 17, 18, 20, 21 or 22, and especially 19. It will be understood by those skilled in the art that maltodextrins with DEs above 20 are referred to as ‘glucose syrups’.

Maltodextrins with DEs above 15 have lower average molecular weights than those with DEs of 15 or below. All maltodextrins are mixtures of polysaccharides with different chain lengths and maltodextrins with DEs above 15 have less of the larger molecular weight sugar units.

We have found that maltodextrins with lower DEs, such as those with a DE of 12 or below, contain longer polysaccharide chains (e.g. with greater than or equal to about 24 glucose units), which have a tendency to form helix structures that may form aggregates when presented in aqueous solutions along with other components, such as active ingredients and/or surfactants, like sucrose esters, giving rise to a turbid solution prior to spray-drying. This turbidity may give rise to stability and/or processability issues during manufacture, requiring the use of in-line filters.

Although we have found that the aforementioned turbidity problem may be alleviated to an extent by reducing the relative amount of maltodextrin that is included within a composition of the invention, which may be achieved by increasing the amount of other ingredients, such as other carrier materials (e.g. disaccharide), the active ingredient or certain additives, such as sucrose esters, the higher the molecular weight of the maltodextrin, the less that needs to be included, and the more e.g. disaccharide or sucrose ester that needs to be added to alleviate the turbidity.

If more sucrose ester is added in order to reduce this turbidity, more may need to be added than is necessary to provide an appropriate (e.g. physical, chemical and/or biological) effect, including an absorption-enhancing effect, as noted herein. Conversely, increasing the amount of disaccharide relative to maltodextrin in the carrier material may have a negative impact on Tg, and therefore the solid-state stability of the composition as noted herein.

We have found that such issues may be reduced, and possibly avoided altogether, by using different maltodextrins altogether, namely those with higher DEs, such as those with a DE above 15, e.g. DE 18, 20 or, more preferably 19.

Notwithstanding the above, polymers, including maltodextrins, that are suitable for use in powder compositions described herein should have a molecular weight that is nevertheless high enough such that, when it is employed in any given amount (in combination with a disaccharide), it is capable of forming a suitable carrier material for the active ingredient, including the provision of an appropriate degree of physical stability.

Mixtures from any of the foregoing lists of disaccharides and/or polymeric materials (including maltodextrins) may be employed.

Amounts of carrier materials that may be employed in compositions of the invention are typically in the range of about 5% to about 99.9%, including up to about 99% (e.g. up to about 95% or about 90%), such as about 10% (e.g. about 25%, including about 35%) to about 85%, including about 50% to about 75%, by weight, based upon the total weight of the composition (whether one dose of said composition is included in the dosing means or otherwise).

Whatever their proportions in the final mixture, compositions of the invention include a spray-dried carrier material that comprises a combination of a disaccharide and a polymeric material (e.g. a dextrin). Thus, the carrier material may be prepared by spray drying the relevant ingredients to form a composite carrier material either prior to spray-drying that carrier material along with the other essential ingredients to form a composition of the invention or, more preferably, is made in situ by spray-drying all of the essential components of the composition of the invention together. Compositions of the invention may be prepared by spray-drying in the presence of a processing aid such as L-leucine. isoleucine, trileucine, L-tyrosine or L-arginine.

Compositions of the invention may comprise at least one biopharmaceutical drug compound. By ‘biopharmaceutical drug compound’, we also include within this definition a biological agent or a biologic, in which the biopharmaceutical drug compound is produced from a living organism or its products, or comprises components of living organisms.

Biopharmaceutical drug compounds include proteins and/or oligo- or polypeptides, enzymes, antibodies/parts thereof, vaccines, nucleotides and the like or antibody analogues, antibody mimetics, immunoglobulins, immunomodulators, or combinations thereof. Biopharmaceutical drug compounds may also include blood (e.g. blood cells suspended in blood plasma), blood components (e.g. human blood components), cells, allergens (e.g. pollen, dust mites, animal dander, mold, medications insect venoms and various foods), genes, viruses (e.g. animal viruses, plant viruses, bacterial viruses, bacteriophage, archaeal viruses, helper virus, mycovirus, neurotropic virus, novel virus, emergent virus, oncovirus, orphan virus, passenger virus, provirus, retrovirus, slow virus, DNA virus, dsDNA virus, dsDNA-RT virus, dsRNA virus, nucleocytoplasmic large DNA viruses (NCLDVs), negative-sense ssRNA virus, RNA virus, ssDNA virus, ssRNA virus, satellite viruses (including single-stranded RNA satellite viruses, double-stranded DNA satellite viruses and single-stranded DNA satellite viruses), virion, viral particles, virus-like particles, capsid virus-like particles, virus components and/or elements (such as capsid, capsomere, endogenous viral element (EVE), nucleocapsid), toxins (e.g. toxins from bacteria (such as anthrax lethal toxin, botulinum toxin, pertussis toxin, staphylococcal enterotoxin B (SEB)), fungi and algae (such as aflatoxins, saxitoxin, neosaxitoxin, amanitin, vomitoxin (deoxynivalenol), diacetoxyscirpenol, T-2 and HT-2 toxins), plants (such as abrin and ricin), venoms (e.g. neurotoxins, hemotoxic, cytotoxic), or combinations thereof. Such biological agents or biological may be used in the treatment or prevention of one of more major indications selected from the group: oncology, cardiovascular diseases, infectious disease, autoimmune or inflammatory diseases, metabolic disease.

It is to be understood that such proteins and/or oligo- or polypeptides that may be employed include those naturally occurring as well as their synthetic analogues, semi-synthetic, synthetic, proteoses. It is preferred that such a protein and/or oligo- or polypeptide is a naturally-occurring, or a recombinant, protein and/or peptide. It is further preferred that such a protein, oligo- and/or polypeptide has a molecular weight of more than 5 kDa, such as more than 10 kDa, for example more than 20 kDa, including more than 30 kDa.

Types of naturally-occurring proteins, oligopeptides and/or polypeptides include cytoskeletal proteins such as actin, Arp2/3, Arp2/3, coronin, dystrophin, formin, FtsZ, gloverin, keratin, myosin, tubulin; extracellular matrix proteins such as collagen, elastin, F-spondin, pikachurin, fibronectin; globular proteins such as plasma proteins (e.g. serum amyloid P component), coagulation factors such as complement proteins (e.g. C1-inhibitor, C3-convertase), factor XIII, protein C, protein S, protein Z, protein Z-related protease inhibitor, thrombin, Von Willebrand Factor, acute phase proteins (e.g. C-reactive protein); hemoproteins (e.g. hemoglobin), cell adhesion proteins (e.g. cadherin, ependymin, integrin, NCAM, selectin), transmembrane transport proteins (e.g. CFTR, Glycophorin D, Scramblase), ion channels (e.g. potassium channels, calcium channels, sodium channels, glucose transporter), hormones and growth factors (e.g. colony-stimulating factors (CSFs), epidermal growth factor (EGF), fibroblast growth factor (FGF), platelet-derived growth factor (PDGF), transforming growth factors (TGFs), vascular endothelial growth factor (VEGF), transmembrane receptors (e.g. rhodopsin), intracellular receptors (e.g. estrogen receptor), DNA-binding proteins (e.g. histones, protamines), transcription and regulation proteins (e.g. CI protein, C-myc, FOXP2, FOXP3, MyoD, P53), RNA-binding proteins (e.g. SRRT), immune system proteins (e.g. immunoglobins, major histocompatibility antigens, T cell receptor), nutrient storage and/or transport proteins (e.g. ferritin), chaperone proteins (e.g. GroEL), cytokines and analogues (including recombinant cytokines), such as IL-1 receptor antagonist, anakinra, IL-4, IL-6, IL-10, IL-12, IL-17, IL-23, IL-27, IL-33, IL-35 or, more particularly, IL-2, IL-7, IL-15, or IL-21, TNF-alfa, IFN-alfa, pifonakin, mobenakin, adargileukin alfa, aldesleukin, celmoleukin, denileukin diftitox, pegaldesleukin, teceleukin, tucotuzumab celmoleukin, daniplestim, muplestim, binetrakin, atexakin alfa, emoctakin, ilodecakin, oprelvekin, edodekin alfa, cintredekin besudotox, iboctadekin, cytokines developed into protein therapeutics (e.g. bone mophogenetic protein (BMP), granulocyte colony-stimulating factor (G-CSF), interferon alfa, IL-11), enzymes (e.g. oxidoreductases, transferases, hydrolases, lyases, isomerases, ligases), proenzymes (e.g. angiotensinogen, trypsinogen, chymotrypsinogen, pepsinogen, prothrombin, plasminogen, procaspases, pacifastin, proelastase, prolipase, procarboxypolypeptidases), such as α-glucosidase, α-D-galactosidase, β-glucocerebrosidase, iduronate-2-sulfatase, n-acetylgalactosamine-6-sulfatase, n-acetylgalactosamine-4-sulfatase, pancreatic enzyme products (PEPs), which contain pancrelipase, mixtures of digestive enzymes, lipase, protease and amylase; alteplase, reteplase, and tenecteplase; dornase alfa, pegloticase, rasburicase, L-asparaginase, collagenase, pegademase (bovine), glucarpidase, ocriplasmin; as well as lactase (from Aspergillus oryzae, Aspergillus niger, Kluyveromyces fragilis, Kluyveromyces lactis or E coli); human peptide hormones such as anti-Müllerian hormone, adiponectin, adrenocorticotropic hormone, angiotensinogen, angiotensin, antidiuretic hormone, atrial natriuretic peptide, cholecystokinin, corticotropin-releasing hormone, cortistatin, endothelin, follicle-stimulating hormone, galanin, gastrin, glucagon-like peptide-1, glucagon and glucagon-like peptide-1 analogues, dasiglucagon, gonadotropin-releasing hormone, growth hormone-releasing hormone, hepcidin, human chorionic gonadotropin, human placental lactogen, growth hormone, inhibin, leptin, lipotropin, luteinizing hormone, melanocyte stimulating hormone, motilin, orexin, osteocalcin, pancreatic polypeptide, prolactin, prolactin-releasing hormone, relaxin, renin, growth hormone-inhibiting hormone, growth hormone release-inhibiting hormone, somatotropin release-inhibiting factor, somatotropin release-inhibiting hormone, thrombopoietin, thyroid-stimulating hormone, thyrotropin, thyrotropin-releasing hormone, guanylin uroguanylin, tetrakosaktid, mecasermin, somapacitan, pegvisomant, desemopressin, terlipressin, lypressin, ornipressin, argipressin, demoxytocin, carbetocin, ocreotide, vapreotide, elkatonin, or combinations thereof.

Other types of types of proteins, oligopeptides and/or polypeptides may include viral or bacterial proteins. The term ‘viral or bacterial protein’ includes proteins which are both a component and a product of the virus or bacterium, and preferably which are encoded by the viral or bacterial genome. Preferably, a virus protein is a component of the capsid or envelope of the virus. The virus protein may be a spike (S) protein of a coronavirus or a portion or variant thereof; the hemagglutinin (H) or neuraminidase (N) proteins of influenza virus, or a portion or variant thereof; the L3 protein of adenovirus, or a portion or variant thereof; a fusion protein of respiratory syncytial virus (RSV), or a portion or variant thereof.

It is preferred that protein, oligopeptide or polypeptide is not human insulin, cyclosporin, insulin, interferon β, interferon γ, TPA, albumin, HGH, factor VIII, erythropoietin, calcitonin, oxytocin, vasopressin, voclosporin, substance P, kassinin, neurokinin A, eledoisin, neurokinin B, VIP (Vasoactive Intestinal Peptide; PHM27), PACAP (Pituitary Adenylate Cyclase Activating Peptide), peptide PHI 27 (Peptide Histidine Isoleucine 27), GHRH 1-24 (Growth Hormone Releasing Hormone 1-24), glucagon, secretin, NPY (NeuroPeptide Y), PYY (Peptide YY), APP (Avian Pancreatic Polypeptide), PPY Pancreatic Polypeptide, proopiomelanocortin (POMC) peptides, endomorphins, enkephalin pentapeptides, prodynorphin peptides, calcitonin, amylin, AGG01, B-type natriuretic peptide (BNP) lactotripeptides, peptidic components from traditional Chinese medicine Colla Corii Asini, buserelin, gonadorelin, goserelin, histrelin, leuprorelin, nafarelin, triptorelin, abarelix, cetrorelix, degarelix, ganirelix, elagolix, relugolix, teverelix, desmopressin, liraglutide, exenatide, lixisenatide, albiglutide, dulaglutide, semaglutide, somatostatin and analogues, octreotide, pasireotide, lanreotide, tenparatide, or peptides that comprise one or more of buserelin, gonadorelin, goserelin, histrelin, leuprorelin, nafarelin, triptorelin, abarelix, cetrorelix, degarelix, ganirelix, elagolix, relugolix, teverelix, leuprolide, liraglutide, octreotide, desmopressin.

Biopharmaceutical drug compounds that may be employed in compositions of the invention also include antibodies. The term ‘antibody’ will be understood to include polyclonal and monoclonal antibodies. The term also includes all isotypes of antibodies, such as: IgG, IgA, IgM, IgD and IgE. Although the antibody may be a polyclonal antibody, it is preferred that it is a monoclonal antibody.

In some circumstances, particularly if the antibody is going to be administered repeatedly to a human patient, it is preferred if the monoclonal antibody is a human monoclonal antibody or a humanised monoclonal antibody. Suitable monoclonal antibodies may be prepared by known techniques, for example those disclosed in ‘Monoclonal Antibodies Meeting the Challenges in Manufacturing, Formulation, Delivery and Stability of Final Drug Product’ Steven Shire, (Woodhead Publishing, 2015), Current Trends in Monoclonal Antibody Development and Manufacturing’, Shire et al (Springer New York, 2010), ‘Therapeutic Monoclonal Antibodies: From Bench to Clinic’, Zhiqiang An (Wiley 2009), ‘Monoclonal Antibodies; A manual of techniques’, H Zola (CRC Press, 1988) and in ‘Monoclonal Hybridoma Antibodies: Techniques and Application’, SGR Hurrell (CRC Press, 1982), the relevant disclosures in which documents are hereby incorporated by reference. Polyclonal antibodies may be produced which are polyspecific or monospecific.

In one embodiment, antibodies may be murine, human (including humanised) or chimeric antibodies. Chimeric antibodies are discussed by Neuberger et al (1998, 8th International Biotechnology Symposium Part 2, 792-799), the relevant disclosure in which document is hereby incorporated by reference. Suitably prepared non-human antibodies can be ‘humanised’ in known ways, for example by inserting the complementarity-determining regions (CDR) of mouse antibodies into the framework of human antibodies. The antibodies may be human antibodies in the sense that they have the amino acid sequence of human antibodies with specificity for the desired antigen or epitope but they may be prepared using methods known in the art that do not require immunisation of humans. For example, transgenic mice are available which contain, in essence, human immunoglobulin genes (see Vaughan et al (1998) Nature Biotechnol. 16, 535-539), the relevant disclosure in which document is are hereby incorporated by reference.

Non-limiting examples of (monoclonal) antibodies which may be used according to the present invention are edrecolomab (L01XC01), tositumomab (V10XA53), rituximab (L01XC02), basiliximab (LO4AC02), infliximab (L04AB02), adalimumab (L04AB04), ibritumomab (V10XX02), vedolizumab (L04AA33), trastuzumab (L01XC03), gemtuzumab ozogamicin (L01XC05), cetuximab (L01XC06), bevacizumab (L01XC07), panitumumab (L01XC08), denosumab (M05BX04), evolocumab (C10AX13), brodalumab (L04AC12), erenumab (N02CD01), catumaxomab (L01XC09), ofatumumab (L01XC10), ipilimumab (L01XC11), erenumab-aooe, brentuximab vedotin (L01XC12), pertuzumab (L01XC13), trastuzumab emtansine (L01XC14), obinutuzumab (L01XC15), dinutuximab beta (L01XC16), nivolumab (L01XC17), natalizumab (L04AA23), ixekizumab (L04AC13), reslizumab (R03DX08), dupilumab (D11AH05), pembrolizumab (L01XC18), blinatumomab (L01XC19), ramucirumab (L01XC21), necitumumab (L01XC22), elotuzumab (L01XC23), daratumumab (L01XC24), mogamulizumab (L01XC25), inotuzumab ozogamicin (L01XC26), olaratumab (L01XC27), durvalumab (L01XC28), bermekimab (L01XC29), avelumab (L01XC31), atezolizumab (L01XC32), cemiplimab (L01XC33), moxetumomab pasudotox (L01XC34), tafasitamab (L01XC35), enfortumab vedotin (L01XC36), polatuzumab vedotin (L01XC37), isatuximab (L01XC38), belantamab mafodotin (L01XC39), dostarlimab (L01XC40), trastuzumab deruxtecan (L01XC41), Bi-specific T-cell Engagers (BiTE; such as Blinatumomab, Solitomab, AMG 330, MT112, MT111, BAY2010112, MEDI-565, fremanezumab, galcanezumab-gnlm, eptinezumab-jjmr, ustekinumab, eculizumab, omalizumab, or combinations thereof.

Other non-limiting examples of antibodies which may be used according to the present invention include etanercept, tocilizumab, siltuximab, sarilumab, olokizumab, sirukumab, tildrakizumab, guselkumab, BI-655066, LY3074828, secukinumab, CNTO6785, bimekizumab, SCH-900117, MLDL1278A, bococizumab, briakinumab, muromonab, abciximab, alemtuzumab, certolizumab, canakinumab, belimumab, idarucizumab, mepolizumab, alirocumab, ocrelizumab, emicizumab, benralizumab, burosumab, lanadelumab, emapalumab, ibalizumab, ravulizumab, romosozumab, risankizumab, brolucizumab, crizanlizumab, sacituzumab, efalizumab, nebacumab, daclizumab, omalizumab, ustekinumab, ado-trastuzumab emtansine, fam-trastuzumab deruxtecan, satralizumab, inebilizumab, teprotumumab, evinacumab, amivantamab, tralokinumab, anifrolumab, loncastuximab tesirine, atoltivimab, maftivimab, odesivimab-ebgn, margetuximab-cmkb, ansuvimab-zykl, aducanumab, aducanumab-avwa, regdanvimab, sotrovimab, tisotumab vedotin, tisotumab vedotin-tftv, tezepelumab, tezepelumab-ekko, tebentafusp, tebentafusp-tebn, faricimab, faricimab-svoa, sutimlimab, sutimlimab-jome, relatlimab, tixagevimab, cilgavimab, casirivimab, imdevimab, tislelizumab, omburtamab, spesolimab, nirsevimab, teclistamab, ublituximab, mosunetuzumab, tremelimumab, penpulimab, lecanemab, inolimomb, teplizumab, donanemab, mirvetuximab soravtansine, toripalimab, sintilimab, retifanlimab, oportuzumab monatox, narsoplimab, or combinations thereof.

Biopharmaceutical drug compounds that may be employed in compositions of the invention also include antibody mimetics. Non-limiting examples of antibody mimetics which may be used include affibody molecules (such as ABY-025), affilins (such as SPVF 2801), affimers, affitins, alphabodies (such as CMPX-1023), anticalins, avimers, designed ankyrin repeat proteins (DARPins such as MP0112), fynomers, kunitz domain peptides (such as Ecallantide (Kalbitor)), adnectins and monobodies (such as Pegdinetanib (Angiocept)), nanoCLAMPs, single domain antibodies such as camelid antibodies, and V_{NAR} fragments obtained from IgNAR, (immunoglobulin new antigen receptor) from cartilaginous fishes, bivalent single-domain antibodies (such as caplacizumab (Cablivi)); and armadillo repeat proteins hereunder designed armadillo repeat proteins, peptide aptamers, and knottins or combinations thereof.

Compositions of the invention in the alternative comprise antibody-derived molecules, such as antibody fragments (F(ab); e.g. ranibizumab (S01LA04)), divalent antibody fragments (F(ab′)₂), as well as related molecules, such as variable fragments (Fv) and other fragments thereof that retain the antigen-binding site, single-chain variable fragments (scFv), (which may be bivalent (e.g. diabody) or trivalent), single domain antibodies (dAbs), or combinations thereof. By ‘bivalent’ we mean that antibodies, antibody mimetics, antibody derived molecules and related molecules have two antigen combining sites. In contrast, monovalent molecules will have only one antigen combining site. A general review of the techniques involved in the synthesis of antibody-derived molecules which retain their specific binding sites is to be found in Winter & Milstein (1991) Nature 349, 293-299.

By ‘ScFv molecules’ we include molecules wherein the V_{H} and V_{L} partner domains are linked via a flexible oligopeptide. The advantages of using antibody-derived molecules, rather than whole antibodies, are several-fold. The smaller size of the fragments may lead to improved pharmacological properties, such as better penetration to the target site. Effector functions of whole antibodies, such as complement binding, are removed. Fab, Fv, ScFv and dAb antibody-derived molecules can all be expressed in and secreted from antibody-derived molecules, thus allowing the facile production of large amounts of molecules.

It is to be understood that such antibodies, antibody mimetics, antibody derived molecules and related molecules may be monospecific, bispecific or polyspecific, or combinations thereof. By bispecific or polyspecific, we include the meaning that they bind two or more different targets.

The term ‘antibodies’ also includes antibody-like molecules which may be screened and selected using both in vivo and in vitro-based selection methods. Such selection methods include yeast surface display, prokaryotic or bacterial surface display, mammalian surface display, ribosome display, mRNA display, cDNA display, CIS display, covalent antibody display (CAD), in vitro compartmentalisation (IVC) and phage-display techniques or other random selection techniques for molecules.

Biopharmaceutical drug compounds that may be employed in compositions of the invention also include the following antibodies/immunoglobulins: immunoglobulins, normal human, for extravascular administration (J06BA01), immunoglobulins, normal human, for intravascular administration (J06BA02), anti-D (rh) immunoglobulin (J06BB01), tetanus immunoglobulin (J06BB02), varicella/zoster immunoglobulin (J06BB03), hepatitis B immunoglobulin (J06BB04), rabies immunoglobulin (J06BB05), rubella immunoglobulin (J066B06), vaccinia immunoglobulin (J06BB07), staphylococcus immunoglobulin (J06BB08), cytomegalovirus immunoglobulin (J06BB09), diphtheria immunoglobulin (J066B10), hepatitis A immunoglobulin (J06BB11), encephalitis, tick borne immunoglobulin (J06BB12), pertussis immunoglobulin (J06BB13), morbilli immunoglobulin (J06BB14), parotitis immunoglobulin (J06BB15), palivizumab (J06BB16), motavizumab (J06BB17), raxibacumab (J06BB18), bezlotoxumab (J06BB21), obiltoxaximab (J06BB22), anthrax immunoglobulin (J061B19), combinations (J06BB30), or combinations thereof.

Compositions of the invention are particularly useful when the biopharmaceutical drug compound comprises one or more vaccines.

Cost is a significant factor when considering improvement to vaccination programs in developing countries. One way to reduce vaccine costs is to generate more stable vaccine compositions, in which such compositions do not require a cold chain, for example, making them cheaper and easier to ship and store. Furthermore, a loss in activity of the vaccine under suboptimal storage conditions is thought to contribute significantly to less effective vaccination programs (Brandau et al, 2003). The World Health Organisation (WHO) requires a minimum titre of vaccine formulations after one week storage at 37° C. However, many vaccines can lose over 50% of their potency when stored for only 1 hour at room temperature (Plotkin & Orenstein, 2004).

In one embodiment, the vaccine can be a live or active vaccine, an attenuated vaccine, a live-attenuated vaccine (e.g. measles, mumps, and rubella (MMR) vaccines, variella (chickenpox) vaccine), an inactivated or killed vaccine (e.g. polio vaccine, influenza vaccine).

In one embodiment, the vaccine may be whole-pathogen vaccine, subunit vaccine or nucleic acid vaccine, such as live-attenuated vaccine, inactivated vaccine, subunit vaccine, recombinant vaccine, polysaccharide vaccine, conjugate vaccine, toxoid vaccine, viral vector vaccine and/or messenger RNA (mRNA) vaccine.

In another embodiment the vaccine can be a subunit vaccine (such as a protein subunit vaccine, e.g. a recombinant vaccine or a virus like particle (VLP) vaccine (including capsid virus-like particle, viral capsid-derived virus-like particle), against e.g. hepatitis B, acellular pertussis vaccines influenza, HIV, HPV, malaria, gonorrhoea, polio, Zika, smallpox, monkey pox, various types of cancer (such as cancers expressing and/or overexpressing HER2 (human epidermal growth factor receptor 2), EGFR (epidermal growth factor receptor), CD-20, VEGF (Vascular endothelial growth factor), VEGFR (Vascular endothelial growth factor receptor), CEA (carcinoembryonic antigen), CA-125 (cancer antigen 125), MUC-1 (mucin 1), or MAGE (melanoma-associated antigen)), more specifically acute respiratory diseases caused by viruses, including respiratory syncytial virus (RSV) or coronaviruses (including variants of either), such as acute respiratory distress syndrome (ARDS) and/or severe acute respiratory syndrome (SARS), and especially COVID-19, or for example, wherein the protein is a spike (S) protein or part thereof; a polysaccharide vaccine, e.g. pneumococcal polysaccharide vaccine, meningococcal vaccine; or conjugate vaccine, e.g. pneumococcal conjugate vaccine, haemophilus influenza type b conjugate, meningococcal conjugate vaccine, MenACWY). The subunit vaccine may be produced in different systems, such as E. coli systems, yeast cell systems, insect cell systems, plant systems, or mammalian cell systems.

In yet another embodiment, the vaccine can be a nucleic acid vaccine (such as a DNA vaccine or an RNA vaccine, preferably wherein the RNA is messenger RNA (mRNA)). Examples of such vaccines may include DNA vaccines against cancer, DNA vaccines against tuberculosis, DNA vaccines against Edwardsiella tarda, DNA vaccines against HIV, DNA vaccines against anthrax, DNA vaccines against influenza, DNA vaccine against dengue, DNA vaccine against typhoid, DNA vaccines against different antigens (e.g. p^{CE6}, p^{CE18}, S iniae DNA vaccine in the form of plasmid pSia10, pIDSia10, pIDOmpU, pSiVa1, etc.), prophylactic DNA vaccines, non-replicating mRNA vaccines, in vivo self-replicating mRNA vaccines, in vitro dendritic cell non-replicating mRNA vaccines, dendritic cell vaccines, personalised cancer vaccines (e.g. where the RNA sequence in the vaccine is designed to code for cancer-specific antigens), RNA vaccine against SARS diseases (such as those caused by the coronavirus SARS-CoV-2, i.e. corona virus 2019 or COVID-19).

In yet another embodiment, the vaccine can be a toxoid vaccine (e.g. tetanus toxoid (TT) vaccine; diphtheria vaccine; diphtheria, pertussis & tetanus (DPT) vaccine; or botulinum toxoid vaccine). For the avoidance of doubt, toxoid vaccines include those using a toxin made by, for example, the germ that causes a disease.

In yet another embodiment, the vaccine can be a viral vector vaccine. The viral vector may be a poxvirus (e.g. vaccina virus, modified vaccinia Ankara (MVA), avipox, fowlpox), a retrovirus (e.g. a lentivirus), vesicular stomatitis virus (VSV), measles virus, an adenovirus, an adeno-associated virus, a cytomegalovirus, Sendai virus, a herpes simplex virus or a combination thereof. The viral vector may be a live or active virus vaccine, an attenuated virus vaccine, a live-attenuated virus vaccine, an inactivated or killed virus vaccine.

In yet another embodiment, the vaccine can be a bacterial vector vaccine. The bacterial vector may be lactic acid bacterium Mycobacterium bovis BCG, Lactococcus lactis, Salmonella, Salmonella spp, Bacillus subtilis, Pseudomonas aeruginosa, Shigella, Shigella spp, Vibrio Cholera, Vibrio anguillarum, Corynebacterium pseudotuberculosis, Bordetella pertussis, Streptococcus, Listeria Monocytogenes, Escherichia coli, Yersinia enterocolitica, Mycobacterium smegmatis, Lactobacillus or a combination thereof.

In yet another embodiment, the vaccine can be a conjugate vaccine, against e.g. pneumococcal bacterial infections.

In yet another embodiment, the vaccine can be a recombinant vector vaccine.

In yet another embodiment, the vaccine can be a nanoparticle-based vaccine (e.g. a mesoporous silica nanoparticle (MSN)-based vaccine or a solid lipid nanoparticle (sLNP)-based vaccine), or a virus like particle (VLP) vaccine (e.g. a capsid VLP vaccine or a viral capsid-derived VLP vaccine), each of which vaccines may be effective against diseases including influenza, HIV, HPV, malaria, gonorrhoea, polio, Zika, smallpox, monkey pox, bacterial infections (such as Chlamydia trachomatis, Neisseria gonorrhoeae, Neisseria meningitidis, Bordetella pertussis, or Mycobacterium tuberculosis), or various types of cancer (such as cancers expressing and/or overexpressing HER2 (human epidermal growth factor receptor 2), EGFR (epidermal growth factor receptor), CD-20, VEGF (Vascular endothelial growth factor), VEGFR (Vascular endothelial growth factor receptor), CEA (carcinoembryonic antigen), CA-125 (cancer antigen 125), MUC-1 (mucin 1), or MAGE (melanoma-associated antigen), colorectal cancer, breast cancer, prostate cancer, lung cancer, cervical cancer and ovarian cancer), more specifically acute respiratory diseases caused by viruses, including respiratory syncytial virus (RSV) or coronaviruses (including variants of either), such as acute respiratory distress syndrome (ARDS) and/or severe acute respiratory syndrome (SARS), and especially COVID-19. The nanoparticle- or VLP-based vaccine may also carry and/or deliver proteins or parts thereof (such as viral, bacterial, mammalian, fungal, or parasitic proteins, which proteins, or parts thereof, may be membrane proteins, surface proteins, fusion proteins, envelope proteins, structural proteins, spike proteins, nucleocapsid or capsid proteins, or combinations thereof), DNA, lipids, polysaccharides, toxins, (such as bacterial toxins), mRNA, RNA, or virus vaccines (such as a live or active virus vaccine, an attenuated virus vaccine, a live-attenuated virus vaccine, an inactivated or killed virus vaccine). The nanoparticle-or VLP-based vaccine may further also carry and/or deliver adjuvants and immune stimulatory molecules, such as TLR agonists, and cytokines.

Optionally, compositions of the invention that are in the form of vaccine compositions may comprise an adjuvant. The term ‘adjuvant’ is intended to mean any compound added to the formulation to increase the biological effect of the one or more products of the invention within the formulation. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be organic polycations (e.g. polyethyleneimine (PEI)), cationic polymers such as cationic cellulose ethers, cationic cellulose esters, deacetylated hyaluronic acid, chitosan, cationic dendnmers, cationic synthetic polymers such as poly(vinyl imidazole), and cationic polypeptides such as polyhistidine, polylysine, polyarginine, and peptides containing these amino acids, cationic (N3) or anionic (13) lipid adjuvants. Depending on the host species, various adjuvants may be used to increase an immunological response. Such adjuvants include, but are not limited to toxoid adjuvants, cytokines, Natural Killer T cell (NKT) ligands (e.g. alpha-Galactosylceramide (alpha GalCer) and analogues), C-type lectin receptor (CLR) ligands, toll-like receptor (TLR) agonists (e.g. CpG, CpG ODN, Poly I:C, glucopyranosyl lipid A (GLA), monophosphoryl lipid A (MPL), resiquimod (R848), flagellin, imidazoquinolines (e.g imiquimod)), STING ligands (e.g. the STING agonist: bis-(3′,5′)-cyclic dimeric guanosine monophosphate (c-di-GMP or cdGMP)), muramyl dipeptide, the ‘Iscoms’ of EP 109 942, EP 180 564 and EP 231 039, surface-active substances such as lysolecithin, polyanions, peptides, limpet hemocyanin (KLH), aluminium salts, alum, Alhydrogel, aluminium hydroxide, aluminium phosphate, saponin-based adjuvants (e.g. Matrix Ml), delta inulin microparticle adjuvants (e.g. Advax), DEAE-dextran, neutral oils (e.g. miglyol), vegetable oils (such as arachis oil), squalene (e.g. shark squalene) emulsions (e.g. MF59, AS03, Freund's complete or incomplete adjuvant, Montanide ISA51, Montanide ISA720), liposomes (e.g. DOPE (1,2-di-(9Z-octadecenoyl)-sn-glycero-3-phosphoethanolamine):DDA (dimethyldioctadecylammonium bromide salt) multilamella liposomes, N-[i-(2,3-Dioleoyloxy) propyl]-N,N,N-trimethylammonium chloride (DOTAP) liposomes), lipid nanoparticles, Poly (lactic-co-glycolic acid) (PLGA) nanoparticles, Pluronic polyols or the Ribi adjuvant system, nucleic acids (e.g. single-stranded RNAs), Hiltonol or a combination thereof. ‘Pluronic’, ‘Alhydrogel’, ‘Hiltonol’, ‘Montanide’ are Registered Trade Marks.

Such vaccines would be suitable for use in vaccinating against, reducing the risk of, preventing, or combatting a disease, disorder and/or condition. Said disease, disorder and/or condition may, for example, be caused by viral, bacterial and/or parasitic infections. Alternatively, said disease, disorder and/or condition may have endogenous and/or genetic/environmental aetiologies, including autoimmune diseases, cardiovascular diseases, inflammatory diseases, metabolic diseases, and/or cancer.

In a preferred embodiment, the virus causing the disease, disorder and/or condition may be an orthomyxovirus (e.g. influenza viruses such as influenza A(H1N1), A(H3N2), A(H7N7), A(H3N8); an isavirus or a thogotovirus), parvovirus (e.g. an adeno-associated-virus, Canine Parvovirus (CPV), Feline Panleukopenia (FPV), mink enteritis virus (MEV)), an adenovirus, a pneumovirus (e.g. a respiratory syncytial virus (RSV)), a herpesvirus (e.g. herpes simplex virus (HSV), feline herpesvirus type-1 (FHV-1), equine herpes viruses (EHV), varicella zoster virus), a matonavirus (e.g. a rubella virus), a rhabdovirus (rabies virus), a retrovirus (e.g. a lentivirus, a human immunodeficiency virus (HIV), a human T-lymphotropic virus (HTLV)), a poxvirus (e.g. a vaccinia virus, a smallpox virus, a monkey pox), a paramyxovirus (e.g. a measles virus, a Newcastle sisease virus, a mumps virus, Canine Distemper Virus (CDV), Canine parainfluenza virus (CPi)), a papillomaviruses (e.g. a human papillomavirus (HPV)), a reovirus (e.g. a rotavirus), a picornavirus (e.g. a poliovirus, a hepatovirus A virus, a hepatovirus B virus), a calicivirus (e.g. Norwalk virus, a sapovirus, a vesivirus such as feline calicivirus (FCV)), a norovirus, a flavivirus (e.g. a West Nile virus; a dengue virus; a tick-borne encephalitis virus, a yellow fever virus, a zika virus, a Japanese encephalitis virus), a togavirus (e.g. alphaviruses) or a coronavirus (e.g. a severe acute respiratory syndrome coronavirus, such as SARS-CoV-1 or SARS-CoV-2).

In a preferred embodiment, the bacteria may be coccus bacteria (e.g. Chlamydia trachomatis, Neisseria gonorrhoeae, Neisseria meningitidis, Staphylococcus aureus, Staphylococcus haemolyticus, Streptococcus agalactiae, Streptococcus bovis, Streptococcus equi, Streptococcus pyogenes), a bacillus bacteria (e.g. Bacillus anthracis, Bordetella pertussis, Bordetella bronchiseptica, Chlamydophila pneumoniae, Clostridium tetani, Corynebacterium diphthenae, Legionella pneumophila, Mycobacterium leprae, Mycobacterium tuberculosis, Vibrio cholerae, Rickettsia), spiral-shaped (spirochete or spirilla) bacteria (e.g. Borrelia burgdorferi, Leptospira, Leptospira interrogans, Treponema pallidum, Treponema denticola), Neorickettsia risticii or mycoplasmas (e.g. Mycoplasma gallisepticum, Mycoplasma hyopneumoniae).

In a preferred embodiment, the parasite may be protozoa (e.g. giardia, plasmodia), helminths (e.g. flatworms, nematodes), or ectoparasites (e.g. ticks, fleas, lice, mites).

In a preferred embodiment, the autoimmune disease may be uveitis, atopic dermatitis, hidradenitis supporativa, systemic lupus erythematosus, rheumatoid arthritis, psoriatic arthritis, multiple sclerosis, type 1 diabetes, myasthenia Gravis, Guillain-Barre syndrome, osteoarthritis, ankylosing spondylitis, Crohn's disease, ulcerative colitis, psoriasis, paroxysmal nocturnal hemoglobinuria, neuromyelitis optica, chronic idiopathic urticaria, migraine or cystic fibrosis.

In one embodiment, the cancer may be a carcinoma, a sarcoma, a lymphoma, a leukaemia, a germ cell cancer or a blastoma. Preferably, the cancer may be bone and muscle cancer, cancers of the brain and nervous system (e.g. glioblastoma, glioma, neuroblastoma, chordoma), breast cancers (e.g. breast cancer, medullary carcinoma, Phyllodes tumors), endocrine system cancers (e.g. thyroid cancer, multiple endocrine neoplasia syndrome, adrenocortical carcinoma), eye cancer (e.g. uveal melanoma, retinoblastoma, optic nerve glioma), gastrointestinal cancers (e.g. gastric (stomach) cancer, colon cancer, rectal cancer, colorectal cancer, pancreatic cancer, liver cancer gallbladder cancer), genitourinary and gynecologic cancers (e.g. bladder cancer, cervical cancer, ovarian cancer, renal cell carcinoma, prostate cancer, nephroblastoma, urothelial bladder cancer), head and neck cancers (e.g. head and neck cancer, esophageal cancer, pharyngeal cancer, oral cancer), hematopoietic cancers (Hodgkin's lymphoma, AIDS-related lymphoma, acute myeloid leukemia, acute lymphoblastic leukemia, non-Hodgkin's lymphoma), skin cancers (e.g. melanoma, basal cell carcinoma, squamous cell skin cancer, keratoacanthoma), thoracic and respiratory cancers (e.g. small cell lung cancer, non-small cell lung cancer, pleuropulmonary blastoma), virus-related cancers (e.g. HIV/AIDS-related cancers).

In another embodiment, the cancer may be expressing and/or overexpressing one or more tumour associated antigens, such as HER2 (human epidermal growth factor receptor 2), EGFR (epidermal growth factor receptor), CD-20, VEGF (Vascular endothelial growth factor), VEGFR (Vascular endothelial growth factor receptor), CEA (carcinoembryonic antigen), CA-125 (cancer antigen 125), MUC-1 (mucin 1), MAGE (melanoma-associated antigen).

Examples of vaccines may thus include injected polio vaccine (Salk vaccine), Hepatitis A vaccine, Rabies vaccine (e.g. canine, feline or human Rabies vaccine), influenza vaccines (e.g. equine influenza vaccines), tick-borne encephalitis vaccine, Eastern and Western equine encephalomyelitis vaccines (EEE/WEE), coronavirus (e.g. SARS-coronaviruses) vaccines (including BBIBP-CorV, CoronaVac, Covaxin, QazVac, TURKOVAC, CoviVac), injected typhoid vaccine, cholera vaccine, plague vaccine, pertussis vaccine, anthrax vaccine, cholera vaccine, plague vaccine, salmonella vaccine, tuberculosis vaccine, typhoid vaccine, enterotoxigenic Escherichia coli vaccine, live attenuated influenza vaccine (LAIV) e.g. FLUMIST, Japanese encephalitis vaccine, measles vaccine, mumps vaccine, measles and rubella (MR) vaccine, measles, mumps, and rubella (MMR) vaccine, measles, mumps, rubella and varicella (MMRV) vaccine, polio vaccine, rotavirus vaccine, rubella vaccine, smallpox vaccine, varicella vaccine, yellow fever vaccine, zoster/shingles vaccine, tick-borne encephalitis vaccine, COVID-19), mRNA vaccines, toxoid vaccines (e.g. tetanus toxoid, diphtheria toxoid, botulinum toxoid), viral vector vaccines, DNA vaccines, recombinant vector vaccines, subunit vaccines (including protein subunit, e.g. hepatitis B, acellular pertussis vaccines, polysaccharide, e.g. pneumococcal polysaccharide vaccine, meningococcal vaccine, or conjugate, e.g. pneumococcal conjugate vaccine, haemophilus influenza type b conjugate vaccine, meningococcal conjugate vaccine), recombinant vaccines, conjugate vaccines, as well as vaccine adjuvants, or combinations thereof.

Other examples of vaccines include ebola vaccines, zika vaccines, poxvirus vaccines (e.g. monkey pox vaccines), RSV vaccines, HIV vaccines, feline Rhinotracheitis (FVR) vaccines, equine West Nile virus vaccines, Potomac Horse Fever (PHF) vaccines, equine herpesvirus vaccines, malaria vaccines (e.g. RTS,S/A S01 or Mosquirix®) or vaccines against cancer (e.g. HEPLISAV-B®, Gardasil®, Cervarix®, T-VEC (Imlygic®), Sipuleucel-T, PROSTVAC®, CVAC-301 (PANVAC®), CV9104, MVA.5T4, MVA-BN(R)-HER2, TroVax®, DCVax-L, TAEK-VAC-HerBy, ChAdOx1-MAGEA3-NYESO, Rindopepimut, and vaccines against HER2 and brachyury-expressing cancers).

In some embodiments, the vaccine may be administered to the subject only once. In other embodiments, the vaccine may be administered to the subject twice or multiple times. For example, in the embodiment in which the vaccine is an inactivated, subunit, recombinant, polysaccharide, conjugate, or toxoid vaccine, it may be appropriate to separately administer a primary dose, and one or more subsequent booster doses, to the subject.

In some embodiment, the vaccine may be administered to the subject in combination to another type of biopharmaceutical drug compound (e.g. antibodies). Thus, immunisation may comprise components of active and/or passive immunisation. Active immunisation provokes active immunity after exposure to an antigen, whereas a passive immunisation provides immunity passively, for example via preformed antibodies produced exogenously (e.g. from another host).

Immunotherapy, optionally wherein the immunotherapy is oncolytic virotherapy. By oncolytic virotherapy (OV), we include the meaning of a form of immunotherapy that uses competent replicating viruses to infect and destroy cancer cells. Preferably, the competent viruses specifically attack tumour cells but not healthy cells.

Compositions of the invention may also comprise:
antigens including exogenous, endogenous and autoantigens, or combinations thereof; and/or
viral vectors including retroviruses (e.g. lentivirus), poxviruses, adenoviruses and adeno-associated virus (AAV)), or combinations thereof.

Biopharmaceutical drug compounds that may be employed in compositions of the invention also include oligonucleotides, including aptamers (including DNA, RNA, XNA or peptide aptamers), morpholinos, CpG oligodeoxynucleotide, polypurine reverse-Hoogsteen hairpins, or combinations thereof; as well as nucleotides (e.g. adenosine monophosphate (AMP), guanosine monophosphate (GMP), cytidine monophosphate (CMP), uridine monophosphate (UMP), cyclic adenosine monophosphate (cAMP), cyclic guanosine monophosphate (cGMP), cyclic cytidine monophosphate (cCMP), cyclic uridine monophosphate (cUMP), deoxyadenosine monophosphate (dAMP), deoxy guanosine monophosphate (dGMP), deoxtcytidine monophosphate (dCMP), (deoxy)thymidine monophosphate (dTMP), adenosine diphosphate (ADP), guanosine diphosphate (GDP), cytidine diphosphate (CDP), uridine diphosphate (UDP), deoxyadenosine diphosphate (dADP), deoxyguanosine diphosphate (dGDP), deoxycytidine diphosphate (dCDP), (deoxy)thymidine diphosphate (dTDP), adenosine triphosphate (ATP), guanosine triphosphate (GTP), cytidine triphosphate (CTP), uridine triphosphate (UTP), deoxyadenosine triphosphate (dATP), deoxyguanosine triphosphate (dGTP), deoxycytidine triphosphate (dCTP), (deoxy)thymidine triphosphate (dTTP)), nucleosides and corresponding nucleobases (e.g. adenine, adenosine, deoxyadenosine, guanine, guanosine, deoxyguanosine, thymine, 5-methyluridine, thymidine, uracil, uridine, deoxyuridine, cytosine, cytidine, deoxycytidine), antisense elements (e.g. antisense oligonucleotides, antisense RNA), siRNA therapeutics (e.g. ONPATTRO® (patisiran) and GIVLAARI™ (givosiran)), mRNA therapeutics, DNA therapeutics, or combinations thereof.

Biopharmaceutical drug compounds that may be employed in compositions of the invention also include immunomodulators, such as interleukins (e.g. IL-2, IL-7, IL-12, IL-27, IL-4, IL-10, IL-35, IL-33), cytokines (e.g. interferons, G-CSF), chemokines (e.g. CCL3, CCL26, CXCL7), cytosine phosphate-guanosine, oligodeoxynucleotides, glucans, or combinations thereof.

In addition, and/or in the alternative, preferred biopharmaceutical drug compounds that may be employed in compositions of the invention include those (listed above or otherwise) with an aqueous solubility that is at least about 10 mg/mL, such as at least about 1 mg/mL, including at least about 100 μg/mL, such as at least about 10 μg/mL, for example at least about 1 μg/mL and, particularly, at least about 0.5 μg/mL, for example at least about 0.1 μg/mL, such as at least about 0.05 μg/mL and particularly at least about 0.01 μg/mL, at room temperature and atmospheric pressure. ‘Aqueous’ solubility will be understood to include not only solubility in pure water, but also in relevant physiological fluids, and especially those found in the nose (which can also be simulated in terms of isotonicity and pH).

Preferred active ingredients that may be employed in compositions of the invention include the fusion protein abatacept and the following antibodies: pembrolizumab, adalimumab, ustekinumab, trastuzumab, bevacizumab, rituximab, nivolumab, infliximab, eculizumab, omalizumab, cetuximab and panitumumab, fremanezumab, galcanezumab-gnlm, and eptinezumab-jjmr; the following enzymes: alteplase reteplase, tenecteplase, dornase alfa, pegloticase, rasburicase and L-asparaginase; and the following vaccines: typhoid vaccines, pertussis vaccines, cholera vaccines, tuberculosis vaccines, typhoid vaccines, measles, mumps and/or rubella (including MMR) vaccines, polio vaccines, yellow fever vaccines, zoster/shingles vaccines, tetanus toxoid, diphtheria toxoid and botulinum toxoid vaccines, and especially influenza vaccines and SARS-coronavirus (e.g. SARS-CoV-2) vaccines.

Other active ingredients that may be employed in compositions of the invention include carcinoembryonic antigen (CEA), cancer-related tumour markers (such as cancer antigen 125 (CA-125)), Mucin 1 (MUC-1), melanoma-associated antigens (MAGE).

Combinations of one or more of the aforementioned active ingredients in the same, or in different classes may be employed.

When compositions of the invention are made by a solvent-based process, as described hereinbefore, including by way of a process of spray-drying, this may result in the presence of active ingredient in a form in which it is no longer in the form of a crystalline salt because it is freely dispersed within, and encapsulated by, the carrier materials in an amorphous form. Compositions of the invention may provide for little to no loss in chemical stability of that active ingredient under the normal storage conditions mentioned herein.

Moreover, when prepared in this way, the biopharmaceutical drug compound that is present in a composition of the invention may possess essentially the same biological activity when compared to the biopharmaceutical drug compound in isolated form, prior to manufacture of the relevant composition. By ‘essentially the same biological activity’, we include that there is less than about 95%, such as less than about 90%, including less than about 85%, less than about 80%, less than about 75%, less than about 70%, less than about 65%, less than about 60%, less than about 55%, less than about 50%, less than about 45%, less than about 40%, less than about 35%, less than about 20%, less than about 25%, less than about 20% or about 15%, and particularly less than about 10% loss in biological activity (which, depending on the biopharmaceutical drug compound that is employed, may include potency, binding activity and/or a pharmacological effect, such as an immune response), as may be measured by way of an appropriate assay for the biopharmaceutical drug compound in question, for example as described hereinafter.

Furthermore, compositions of the invention may be stored in isolated solid form, when formulated into a pharmaceutical formulation or dosage form, and/or when loaded into a pharmaceutical dosing means, such as a nasal applicator or a reservoir therefor (with or without appropriate pharmaceutical packaging) or otherwise, under normal storage conditions as hereinbefore defined, with an insignificant degree of loss in biological activity of the biopharmaceutical drug compound (e.g. less than about 95%, such as less than about 90%, including less than about 85%, less than about 80%, less than about 75%, less than about 70%, less than about 65%, less than about 60%, less than about 55%, less than about 50%, less than about 45%, less than about 40%, less than about 35%, less than about 20%, less than about 25%, less than about 20% or about 15%, and particularly less than about 10% of loss in biological activity of the biopharmaceutical drug compound), as may be measured by appropriate assay for the biopharmaceutical drug compound in question, for example as described hereinafter.

The amount of active ingredient that may be employed in a single dose of a composition of the invention must be sufficient so exert its pharmacological effect. For transmucosally- (e.g. sublingually-, buccally- and, particularly, intranasally-) administered compositions of the invention, that amount must not exceed about 100 mg in a single dose. Actual doses of the relevant biopharmaceutical drug compounds mentioned above include those that are known in the art and may be described for the active ingredients in question to in the medical literature, such as Martindale—The Complete Drug Reference, 40^{th} Edition, Pharmaceutical Press, London (2020) and the documents referred to therein, the relevant disclosures in all of which documents are hereby incorporated by reference. However, compositions of the invention may be found to exhibit good bioavailability and/or rapid absorption, resulting in a more rapid onset of action and/or higher plasma concentrations, compared to prior art compositions comprising the same active ingredient.

In this respect, pharmacologically-appropriate amounts of active ingredients in compositions of the invention may be less than those referred to in the literature (vide supra). Such amounts may nevertheless be determined by the skilled person and may vary with the type and severity of the condition that is to be treated, and what will be most suitable for an individual patient. This is also likely to vary with the nature of the formulation, as well as the type and severity of the condition that is to be treated, as well as the age, weight, sex, renal function, hepatic function and response of the particular patient to be treated.

Depending upon the potency of the biopharmaceutical drug compound, and upon the final dosage form that is to be employed, the total amount of active ingredient that may be employed in a composition of the invention may be in the range of about 0.0001%, or about 0.0002% for example about 0.001%, such as about 0.01%, including about 0.1%, (e.g. about 1%, about 2% or about 5%), such as about 10% (e.g. about 20%) up to about 95%, such as about 75%, for example about 50%, e.g. about 40%, by weight based upon the total weight of the composition. This is independent of the number of separate doses of composition (which should be the same) that are initially present in a dosing means according to the invention.

For transmucosal, including pulmonary, buccal, sublingual or, preferably, intranasal, administration, appropriate doses of active ingredients (calculated as the free acid/base) per unit dosage are in the range of about 0.01 μg, such as about 0.1 μg, including about 1 μg (e.g. about 10 μg, such as about 250 μg) up to about 100 mg (e.g. about 80 mg), such as between about 1 mg and about 60 mg (e.g. about 3 mg, such as about 10 mg to about 50 mg), depending on the active ingredient that is employed.

For other forms of administration (e.g. administration by injection or perorally), appropriate doses of active ingredients (calculated as the free acid/base) per unit dosage are in the range of about 1 μg to about 1,000 mg, such as about 500 mg (e.g. about 400 mg), such as between about 1 mg and about 300 mg (e.g. about 3 mg, such as about 10 mg to about 200 mg), depending on the active ingredient that is employed.

Alternatively, the appropriate dose of the active ingredients can be based on the biological activity or effect (rather than the mass of the biopharmaceutical compound) of the different biological agents (i.e. proteins, oligopeptides, polypeptides, enzymes, antibodies and parts thereof, vaccines, nucleotides and the like, antibody analogues, antibody mimetics, immunoglobulins, immunomodulators, blood, blood components, cells, allergens, genes, viruses, toxins, venoms or combinations thereof). Appropriate doses may be in the range of about 10⁻⁹ international unit (IU) to about 1×10⁹ IU. By international unit (IU), we include the meaning of a quantity of active ingredient that produces a specified effect when tested according to internationally accepted standardised biological procedures (see World Health Organization (WHO) International Standards). Methods for calculating the appropriate IU depending on the different biopharmaceutical compound will be known to those skilled in the art.

According to three further aspects of the invention there is provided:
a composition of the invention for use in the treatment of a condition for which the at least one biopharmaceutical drug compound that is/are included therein is/are useful for (for example by transmucosal, such as intranasal, administration of said composition);
the use of a composition of the invention for the manufacture of an (e.g. transmucosal, such as an intranasal) medicament for the treatment of a condition for which the at least one biopharmaceutical drug compound that is/are included therein is/are useful for; and
a method of treatment of a condition for which the at least one biopharmaceutical drug compound that is/are included within composition of the invention is/are useful for, which method comprises the (e.g. transmucosal, such as intranasal) administration of a composition of the invention to a patient suffering from, or susceptible to, said condition.

Compositions of the invention are useful, depending on the nature of the active ingredient(s) that is/are included in such a composition, in the treatment of a wide range of clinical conditions including rheumatoid arthritis, psoriasis, ankylosing spondylitis, Crohn's disease, multiple sclerosis, diabetic retinopathy, age-related macular degeneration, diabetes mellitus, diabetes insipidus, cancers, as well as psoriatic arthritis and chronic obstructive pulmonary disease (COPD).

Compositions of the invention comprising e.g. abatacept and adalimumab (an anti-TNF(alpha) agent) may be used in the treatment of rheumatoid arthritis, psoriatic arthritis, ankylosing spondylitis, Crohn's disease, ulcerative colitis, psoriasis, hidradenitis suppurativa, uveitis, and juvenile idiopathic arthritis; compositions of the invention comprising e.g. ranibizumab and aflibercept may be used in the treatment of diabetic retinopathy and age-related macular degeneration; compositions of the invention comprising e.g. adalimumab may be used in the treatment of psoriasis and ankylosing spondylitis; compositions of the invention comprising e.g. cetuximab and panitumumab may be used in the treatment of EGFR-expressing metastatic colorectal carcinoma; compositions of the invention comprising pembrolizumab (an anti-PD-1 agent) may be used to treat melanoma, non-small cell lung cancer, head and neck cancer, Hodgkin's lymphoma, stomach cancer, cervical cancer, urothelial cancer, colorectal cancer, renal cell carcinoma and breast cancer; compositions of the invention comprising ustekinumab (an anti-interleukin (IL)-12/23 agent) may be used to treat psoriasis; compositions of the invention comprising trastuzumab (an anti-HER2 agent may be used to treat breast cancer; compositions of the invention comprising bevacizumab (an anti-VEGF agent) may be used to treat colon cancer; compositions of the invention comprising rituximab (an anti-CD20 agent) may be used to treat non-Hodgkin's lymphoma; compositions of the invention comprising nivolumab (an anti-PD1 agent) may be used to treat melanoma, non-small cell lung cancer, renal cell carcinoma and head and neck cancer; compositions of the invention comprising infliximab (an anti-TNF(alpha) agent) may be used to treat rheumatoid arthritis, psoriatic arthritis, ankylosing spondylitis, Crohn's disease, ulcerative colitis and psoriasis; compositions of the invention comprising eculizumab (an anti-complement component C5 agent) may be used to treat paroxysmal nocturnal hemoglobinuria (PNH), atypical hemolytic uremic syndrome (aHUS) and neuromyelitis optica; compositions of the invention comprising omalizumab (an anti-IgE agent) may be used to treat asthma and chronic idiopathic urticaria; compositions of the invention comprising cetuximab and panitumumab (anti-EGFR agents) may be used to treat colorectal cancer; compositions of the invention comprising fremanezumab (e.g. fremanezumab-vfrm), galcanezumab (e.g. galcanezumab-gnlm), and eptinezumab (e.g. eptinezumab-jjmr) (calcitonin gene-related peptide (CGRP) inhibitors) may be used to treat migraine.

Compositions of the invention comprising relevant enzymes may be used in a variety of conditions such as glycogen storage disorders (α-glucosidase), lipid storage disorders (α-D-galactosidase A and β-glucocerebrosidase), mucopolysacchandoses (α-L-iduronidase and iduronate-2-sulfatase), mucopolysaccharidoses (N-acetylgalactosamine-6-sulfatase and N-acetylgalactosamine-4-sulfatase), various pancreatic disorders, including cystic fibrosis, Shwachman-Diamond syndrome, chronic pancreatitis, pancreatic tumors, or removal of all or a part of the pancreas (PEPs), acute myocardial infarction (alteplase, reteplase, and tenecteplase), cystic fibrosis (dornase alfa), chronic gout (pegloticase), tumor lysis syndrome (rasburicase), leukemia (L-asparaginase), collagen-based disorders such as Dupuytren's contracture (collagenase), severe combined immunodeficiency disease (pegademase, bovine), detoxification of methotrexate (glucarpidase), vitreomacular adhesion (ocriplasmin), acute myocardial infarction (alteplase, reteplase and tenecteplase), cystic fibrosis (dornase alfa), chronic gout (pegloticase), tumor lysis syndrome (rasbuncase) and L-leukemia (asparaginase).

Compositions of the invention comprising vaccines may be used to treat the relevant condition within which it is intended to provoke an immune response, including any of those described hereinbefore.

As mentioned hereinbefore, compositions of the invention may also include, or may also be administered along with, one or more alkyl saccharides. Compositions of the invention that comprise alkyl saccharides may be found to exhibit surprisingly good bioavailability and speed of absorption compared to corresponding compositions that do not include, for example, alkyl saccharides, and/or include different excipients that are known to act as surfactants.

Alkyl saccharides that may be employed include alkyl glycosides, which may be defined as any sugar joined by a linkage to an alkyl group, such as a C₇₋₁₈ alkyl glycoside. Alkyl glycosides thus may include alkyl maltosides (such as dodecyl maltoside), alkyl glucosides, alkyl sucrosides, alkyl thiomaltosides, alkyl thioglucosides, alkyl thiosucroses and alkyl maltotriosides. However, we prefer that the alkyl saccharide is a sugar ester.

Sugar esters that may be used in the compositions of the invention include trisaccharide esters, such as raffinose esters, monosaccharide esters, such as glucose esters, galactose esters and fructose esters, and/or, preferably, disaccharide esters, such as maltose esters, lactose esters, trehalose esters and, in particular, one or more sucrose esters.

Sucrose esters that may be employed in compositions of the invention have a hydrophilic-lipophilic balance value of between 6 and 20. The term ‘hydrophilic-lipophilic balance’ (HLB) is a term of art that will be well understood by those skilled in the art (see, for example, ‘The HLB System: A Time-Saving Guide to Emulsifier Selection’, published by ICI Americas Inc, 1976 (revised 1980), in which document, Chapter 7 (pages 20-21) provides a method of how to determine HLB values). The longer the fatty acid chains in the sucrose esters and the higher the degree of esterification, the lower the HLB value. Preferred HLB values are between 10 and 20, more preferably between 12 and 20.

Sucrose esters thus include C₈₋₂₂ saturated or unsaturated fatty acid esters, preferably saturated fatty acid esters and preferably C₁₀₋₁₈ fatty acid esters and most preferably C₁₂ fatty acid esters. Particularly suitable fatty acids from which such sucrose esters may be formed include erucic acid, behenic acid, oleic acid, stearic acid, palmitic acid, myristic acid and lauric acid. A particularly preferred such fatty acid is lauric acid. Commercially-available sucrose esters include those sold under the trademark Surfhope® and Ryoto® (Mitsubishi-Kagaku Foods Corporation, Japan).

Sucrose esters may be diesters or monoesters of fatty acids, preferably monoesters, such as sucrose monolaurate. The skilled person will appreciate that the term ‘monolaurate’ refers to a mono-ester of lauric acid, and that the terms ‘lauric acid ester’ and ‘laurate’ have the same meaning and can therefore be used interchangeably. Commercially available sucrose monolaurate products are also sometimes referred to as ‘sucrose laurate’. Commercially-available sucrose monolaurate (or sucrose laurate) products, such as Surfhope® D-1216 (Mitsubishi-Kagaku Foods Corporation, Japan), which may contain small amounts of diesters and/or higher sucrose esters, and minor amounts of other sucrose esters and free sucrose, are suitable for use in the invention. The skilled person will understand that any reference to a specific sucrose ester herein includes commercially available products comprising that sucrose ester as a principal component.

Preferred sucrose esters contain only one sucrose ester, which means that a single sucrose ester (e.g. a commercially-available sucrose ester product) contains a single sucrose ester as the/a principal component (commercially available products may contain impurities, for example a monoester product may contain small amounts of diesters and/or higher esters, such products may be considered to ‘contain only one sucrose ester’ in the context of the present invention). As used herein, the term ‘principal component’ will be understood to refer to the major component (e.g. greater than about 50%, such as about 70% weight/weight or volume/volume) in a mixture of sucrose esters, such as commonly commercially available surfactant products, which are typically sold with a certain range of ester compositions.

A particularly preferred sucrose ester is sucrose monolaurate.

Whether included within a composition of the invention, or in a final dosage form including one or more compositions of the invention, amounts of alkyl saccharide that may be employed may be in the range of about 0.1% to about 10%, such as about 0.5% to about 5%, preferably about 0.75% to about 3% (e.g. to about 2%, such as about 1%), by weight, based upon the total weight of the composition.

Further, optional, additional excipients may be employed within, or administered along with, compositions of the invention, including one or more (further) surfactants. Surfactants that may be mentioned include polyoxyethylene esters (e.g. Myrj™), including polyoxyl 8 stearate (Myrj™ S8), polyoxyl 32 stearate (Gelucire® 48/16), polyoxyl 40 stearate (Myrj™ S40), polyoxyl 100 stearate (Myrj™ S100), and polyoxyl 15 hydroxystearate (Kolliphor® HS 15), polyoxyethylene alkyl ethers (e.g. Brij™), including polyoxyl cetostearyl ether (e.g. Brij™ CS12, CS20 and CS25), polyoxyl lauryl ether (e.g. Brij™ L9 and L23), and polyoxyl stearyl ether (e.g. Brij™ S10 and S20), and polyoxylglycerides (e.g. Gelucire®), including lauroyl polyoxylglycerides (Gelucire® 44/14) and stearoyl polyoxylglycerides (Gelucire® 50/13), sorbitan esters (e.g. Span™), including sorbitan monopalmitate (Span™ 40) and sorbitan monostearate (Span™ 60), polysorbates (Tweens™), including polysorbate 40 (polyoxyethylene (20) sorbitan monopalmitate), polysorbate 60 (polyoxyethylene (20) sorbitan monostearate) and polysorbate 20 (polyoxyethylene (20) sorbitan monolaurate), and sodium lauryl sulfate; and monoacyl glycerols (monoglycerides), such as 2-oleoylglycerol, 2-arachidonoylglycerol, monolaurin, glycerol monomyristate, glycerol monopalmitate, glyceryl hydroxystearate and, preferably, glycerol monostearate, glycerol monooleate (e.g. Cithrol®) and glycerol monocaprylate (e.g. Capmul®). Other surfactants may include lauryl lactate, dipalmitoylphosphatidylcholine (DPPC) and poloxamers.

Other optional additional ingredients (excipients) that may be included within, or administered along with, compositions of the invention, include isotonicity and/or osmotic agents (e.g. sodium chloride), sterols (or steroid alcohols), such as cholesterol and phytosterols (e.g. campesterol, sitosterol, and stigmasterol); antioxidants (e.g. sodium metabisulfite or, in addition, α-tocopherol, ascorbic acid, potassium ascorbate, sodium ascorbate, ascorbyl palmitate, butylated hydroxytoluene, butylated hydroxyanisole, dodecyl gallate, octyl gallate, propyl gallate, ethyl oleate, monothioglycerol, vitamin E polyethylene glycol succinate, or thymol); chelating (complexing) agents (e.g. edetic acid (EDTA), citric acid, tartaric acid, malic acid, maltol and galactose, including salt forms of any of these agents); preservatives (e.g. benzalkonium chloride or, in addition, benzyl alcohol, boric acid, parabens, propionic acid, phenol, cresol, or xylitol); viscosity modifying agents or gelling agents (such as cellulose derivatives, including hydroxypropylcellulose, methylcellulose, hydroxypropyl methylcellulose, carboxymethylcellulose, etc., starches and modified starches, colloidal silicon dioxide, aluminium metasilicate, polycarbophils (e.g. Noveon®), carbomers (e.g. Carbopol®) and polyvinylpyrrolidone); mucoadhesive polymers, such as carboxymethyl cellulose, modified cellulose gum and sodium carboxymethyl cellulose (NaCMC); starch derivatives, such as moderately cross-linked starch, modified starch and sodium starch glycolate; crosslinked polyvinyl pyrollidone, acrylic polymers, such as carbomer and its derivatives (Polycarbophyl, Carbopol®, etc.); polyethylene oxide (PEO); chitosan (poly-(D-glucosamine)); natural polymers, such as gelatin, sodium alginate, pectin; scleroglucan; xanthan gum; guar gum; poly co-(methylvinyl ether/maleic anhydride); and croscarmellose (e.g. croscarmellose sodium); pH buffering agents (e.g. citric acid, maleic acid, malic acid, or glycine, or corresponding salts thereof, such as sodium citrate); colouring agents; penetration enhancers (e.g. isopropyl myristate, isopropyl palmitate, pyrrolidone, or tricaprylin); other lipids (neutral and polar); aromatic carboxylic acids, such as benzoic acid optionally substituted with one or more groups selected from methyl, hydroxyl, amino, and/or nitro, for instance, toluic acid or salicylic acid; and, if appropriate, flavourings (e.g. lemon, peppermint powder or, preferably, menthol), sweeteners (e.g. neohesperidin, acesulfame K or, preferably, sucralose) and dyestuffs. Other excipients may include trisaccharides (e.g. raffinose) and mannitol, as well as pH adjusting agents (e.g. hydrochloric acid and sodium hydroxide).

Total amounts of such ‘additional’ excipients (including surfactants that are not an alkyl saccharide that may be present in compositions of the invention) that may be included along with a composition of the invention per se (irrespective of the dosage form it is included in) may also be up to about 15% (e.g. about 10%), such as up to about 5%, by weight, based on the total weight of the composition.

Total amounts of such ‘additional’ excipients that may be included within a final dosage form including one or more compositions of the invention, may be up to about 99.99%, such as up to about 99.9%, including up to about 99%, for example up to about 90%, for example if the one or more additional excipients is a filler or a carrier in a tablet, a film or the like.

The skilled person will appreciate that, if any additional optional ingredients are included within compositions of the invention, the nature of those ingredients, and/or the amounts of those ingredients that are included, should not have a detrimental effect on the Tg of the composition for the reasons described hereinbefore. In this respect, such optional ingredients may be incorporated in the spray-drying process (i.e. mixed together along with the active ingredient and the carrier materials in the appropriate volatile solvent and then spray-dried), or may be included separately to the spray-dried plurality of particles.

According to a further aspect of the invention, there is provided the compositions of the invention for use in medicine (human and veterinary), and thus in the treatment of patients in need of medical treatment of a condition that the relevant active ingredient is known to treat.

By ‘treatment’ of such conditions, we include the prophylaxis or the diagnosis of such conditions, in addition to therapeutic, symptomatic and palliative treatment.

Compositions of the invention may be administered by any suitable dosing means that is known to the skilled person. Compositions of the invention may be administered transmucosally, and in particular intranasally, by way of a suitable nasal applicator, or a dispenser, means, which means is capable of administering a suitable dose of active ingredient in the form of one or more compositions of the invention to the nasal cavity. A suitable nasal dosing means and/or applicator should thus be capable of housing, and storing, the one or more doses of the relevant composition of the invention itself, or capable of being attached to a reservoir/container that houses and stores the one or more doses of the composition of the invention, and to do so without the consequence of a significant loss of physical and chemical integrity of the composition, including by way of ingress of water. In this way, the composition will be usable as soon as the applicator device is actuated by an end user (whether this is single dose or multiple dose usage), whereupon the applicator will deliver composition (e.g. powder) with an appropriate dose of active ingredient as defined herein to the nasal mucosa of a subject.

Appropriate applicator means have been described in the prior art. When used with compositions of the invention, such compositions may be loaded into a reservoir that is attached to, or forms part of, such an applicator means, whereupon it is contained until the applicator means, or dispenser, is actuated. Hereinafter the terms ‘applicator’, ‘dispenser’, ‘device’ ‘applicator means’, ‘dispensing means’, ‘applicator device’, ‘dispensing device’ and ‘insufflator’ may be used interchangeably and mean the same thing.

The reservoir that contains the solid, multi-particulate powder composition of the invention may be opaque. Because of the stability of compositions of the invention, there is no need to inspect the contents of the reservoir (i.e. the powder composition) prior to administration or use.

The term ‘opaque’ will be understood by those skilled in the art to include ‘not transparent or translucent, impenetrable to light, and/or not allowing light to pass through’.

Applicators comprising compositions of the invention therefore do not (or do not need to) include an inspection window through which the contents of the reservoir of an applicator can be observed and may, in this respect, be wholly opaque in its character, that is at least about 98%, such as at least about 99%, and particularly about 99.9% opaque, and/or no more than about 2%, such as no more than about 1% and particularly about 0.1% transparent, translucent and/or penetrable to light, to allow for inspection of reservoir's contents.

Such applicator means may thus also include a mechanism for expelling the powder composition as described herein from the reservoir through an exit means, which exit means includes anything sized for placement within a human body cavity, such as a nostril, such as an appropriately-shaped nozzle.

The mechanism for expelling the powder may thus include a means for actuating the device, which may include breath-activated actuation or an actuating means for generating a force upon actuation of the device by a user.

Thus, the applicator should be capable of providing a reproducible and sufficient amount of powder composition in a single administration step (and in a manner in which the device does not require ‘priming’), that will provide a therapeutic dose of active ingredient.

Nasal applicators/inhalation devices that may be employed to administer compositions of the invention in the form of powders may include multiple-dose applications, such as metered dose inhalation devices (MDIs), dry powder inhalation devices (DPIs; including low, medium and high resistant DPIs) and soft mist inhalation devices (SMIs) that may be adapted based on technology that is known in the field of delivery of active ingredients to the lung.

In MDIs, compositions of the invention should be capable of forming a stable suspension when suspended in solvents that are typically employed therein, such as a propellant, which propellant has a sufficient vapour pressure to form aerosols upon activation of the delivery device (e.g. a hydrocarbon, a fluorocarbon, a hydrogen-containing fluorocarbon, or a mixture thereof).

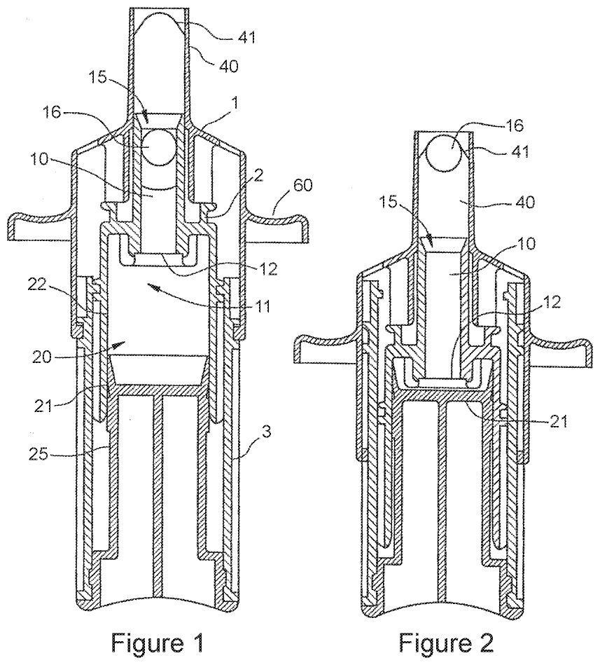

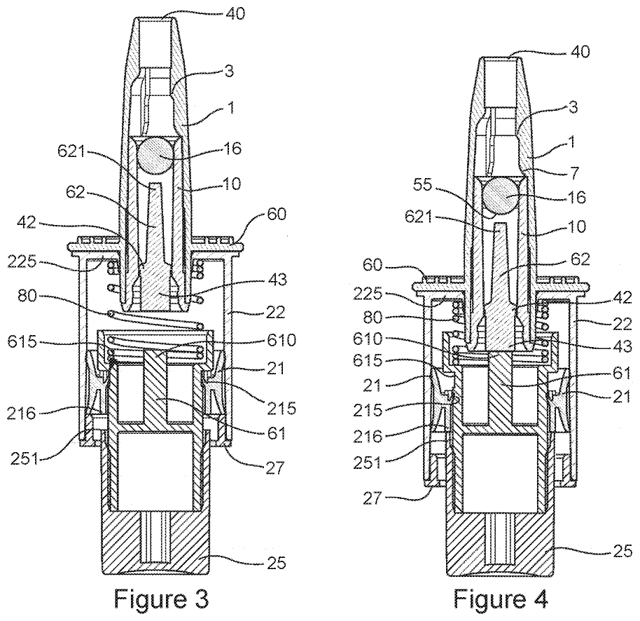

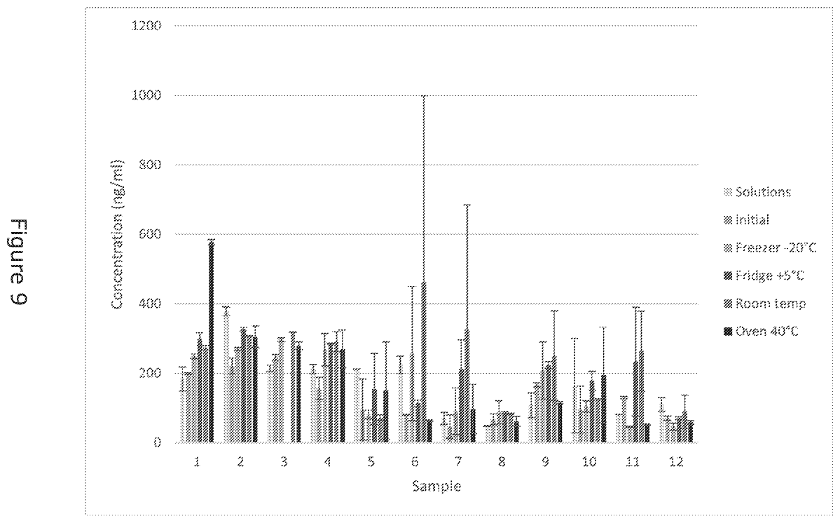

However, if the nasal applicator is a single dose applicator from which a composition is dispensed following actuation, and is then disposed of after use, suitable applicator means or devices for delivering single doses of active ingredients include breath-assisted and blow-assisted devised (such as the Optinose®), as well as those described in U.S. Pat. No. 6,398,074, 6,938,798 or 9,724,713, the relevant disclosures in all of which documents are incorporated herein by reference. FIGS. 1 and 2 of the present application are based on FIG. 1 and FIG. 2, respectively, of U.S. Pat. No. 6,398,074, and FIGS. 3 to 7 are based on FIG. 19 to FIG. 23, respectively, of U.S. Pat. No. 9,724,713. Both are illustrations of applicators that may be employed to administer a composition of the invention intranasally.

In FIG. 1, the device comprises an upper body/dispenser head 1 incorporating an outlet channel 40 (i.e. part of the ‘exit means’ as hereinbefore described) and a gripping means 60 allowing the user to actuate the device. Inside the upper body/dispenser head 1 an element is mounted, designated in its assembly by reference number 2, that incorporates a reservoir 10 and an air chamber 22 for the air blast 20. It is possible for this element 2 to be produced in one piece with the body 1. A lower body 3 is also provided in order to be able to slide relative to the upper body 1 and relative to the element 2, the user exerting a push force on the lower body to actuate the device.

The reservoir 10 contains a single dose of a composition of the invention. The reservoir 10 has an air inlet 11 and a product outlet 15. A product retention device 12, comprising a grid that is permeable to air, is disposed in the air inlet 11 to keep the product in the reservoir 10 until the composition is dispensed. The product outlet 15 is blocked, preferably in a sealed fashion, by a closing ball 16, which is removed from its blocking position by the flow of air when the applicator is actuated and the product is being dispensed.

When a user actuates the device, a pressure is exerted on the plunger 25 in such a way that the piston 21 compresses the air 20 contained in the chamber 22. Since the grid 12 is permeable to air, the compression of the air in chamber 22 creates a blast of air that is transmitted to the reservoir 10 and consequently is applied to the closing ball 16 which is blocking the product outlet 15.

The dimensions of the closing ball 16 and its fixing at the reservoir product outlet 15 are such that the ball 16 is removed from its blocking position, when a minimum predetermined pressure is created through the reservoir 10 by way of a blast of the air 20.

The pre-compression created by the closing ball 16 ensures that when it is removed from its blocking position, the energy accumulated in the hand of the user is such that the piston 21 integral with the plunger 25 is propelled within the chamber 22 thereby creating a powerful blast of air 20, that is to say an air flow suitable to finely spray the dose of composition of the invention.

When this minimum pressure is reached, the ball is quickly moved towards the outlet channel 40 of the device and the flow of air 20 created by the blast expels substantially all of the dose of composition of the invention that is contained within the reservoir 10. Preferably, the outlet channel 40 has a diameter greater than the diameter of the closing ball 16 in order to allow the dose of product to be expelled through the outlet channel 40 by flowing around the ball 16. As shown in FIG. 2, which represents the same device after actuation, the channel 40 comprises a means 41 of arresting or fixing the ball 16 in order to prevent its expulsion out of the device when the product is being expelled.

A further embodiment that may be employed to administer compositions of the invention intranasally is provided in U.S. Pat. No. 9,724,713 at column 7, line 50 to column 8, line 61 and FIGS 19 to 23, which are reproduced as FIGS. 3 to 7 of the present application.

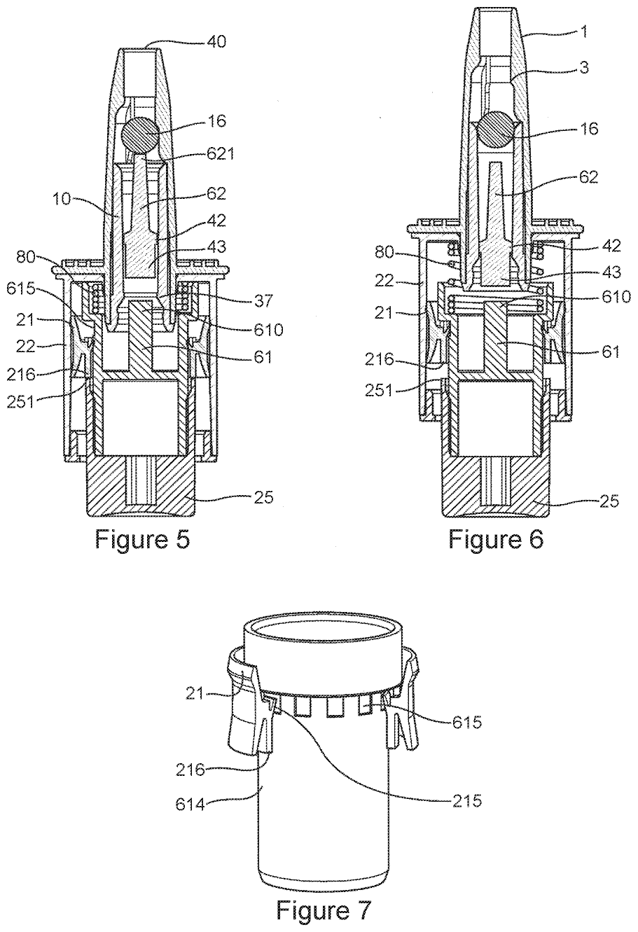

In this embodiment, the reservoir 10 is secured in the upper body/dispenser head 1 which includes the dispenser outlet channel 40 (i.e. part of the ‘exit means’ as hereinbefore described), which has gripping means or finger rest 60, which allows the user to actuate the device. A radial shoulder 37 (see FIG. 5) of the upper body/dispenser head 1 advantageously defines the assembled position of the reservoir 10 in said of the upper body/dispenser head 1.

The mechanical opening system includes a set of rods 61, 62, wherein a second rod portion 62 is pushed by said first rod portion 61 when the device is actuated. At the end of their actuation stroke, i.e. in the dispensing position, the set of rods 61, 62 co-operate with the closure element 16, which is spherical, in particular a ball as in the first embodiment discussed above, so as to expel it mechanically from its closed position.

In this embodiment, the piston 21 is separate from the first rod portion 61, and slides both relative to the air chamber 22 and to a cylindrical surface 614 that is secured to the first rod portion 61. FIG. 7 is a diagrammatic perspective view of the air expeller of the device in FIGS. 3 to 6, in its rest position.

The air chamber 22 may thus be cylindrical, and in its rest position is put into communication with the surrounding air at fluting or grooves 615 that are formed in said cylindrical surface 614 and that co-operate with the piston 21, in particular in its rest position. The piston 21 thus includes an inner lip 215 that slides in airtight manner over the cylindrical wall 614 during actuation, and that co-operates with said fluting 615 in its rest position. The piston 21 also includes an axial extension 216 that co-operates with a top edge 251 of the pusher element 25 (termed a ‘plunger’ in the first embodiment) that moves said piston 21 in the air chamber 22 during actuation.

A retainer member 42 is extended downwards by an axial extension 43 that comes into contact with the top axial end 610 of the first rod portion 61 during actuation.

In addition, in this embodiment, there is no outer body, but merely a cover 27 that is assembled on the bottom axial edge of the air chamber 22.

A spring 80 is provided between the radial flange 225 of the air chamber 22 and the part that forms the first rod portion 61 and the cylindrical surface 614, so as to return the air expeller automatically into its rest position after actuation.

The operating principle is as follows. In the rest position in FIG. 3, the reservoir 10 is closed in sealed manner by the retainer member 42 and by the closure element/ball 16. The air expeller is open to the atmosphere by co-operation between the inner lip 215 of the piston 21 and the fluting 615 of the cylindrical surface 614.

When it is desired to actuate the device, the user presses on the pusher element 25. During this initial stroke, the inner lip 215 of the piston leaves the fluting 615 so as to come to co-operate in airtight manner with the cylindrical surface 614, thereby closing the air chamber 22. At the same moment, the top edge 251 of the pusher element 25 comes into contact with the axial extension 216 of the piston 21, and the top axial end 610 of the first rod portion 61 comes into contact with the axial extension 43 of the retainer member 42.

However, the top axial end 621 of the second rod portion 62 is still not in contact with the rounded surface 55 of the closure element/ball 16, as can be seen in FIG. 4.

Continued actuation thus simultaneously moves the piston 21 in the air chamber, thereby compressing the air contained therein, and moves the retainer member 42 away from its position of closing the reservoir 10. When the second rod portion 62 contacts the rounded surface 55 of the closure element/ball 16, said closure element/ball is expelled mechanically from its closed position, so as to enable the composition to be expelled under the effect of the air compressed by the air expeller.

The dispensing position is shown in FIG. 5. As can be seen in FIG. 5, the retainer member 42 may become detached from the first rod portion 61 while the composition is being expelled under the effect of the compressed air provided by the air expeller. In this position, said closure element/ball is expelled out from the reservoir 10 so as to enable the fluid or powder to be dispensed under the effect of the compressed air. The closure element/ball 16 thus becomes jammed in splines 3 of the upper body/dispenser head 1, which splines prevent in particular any risk of said closure element/ball 16 being expelled out from said upper body dispenser head 1.

When the user relaxes the device, as shown in FIG. 6, the spring 80 that was compressed during actuation, returns the first rod portion 61 towards its rest position. This creates suction that sucks the closure element 16 and the retainer member 42 back towards, or close to, their closure positions. This thus blocks the path for new suction so as to avoid soiling the air expeller while it returns automatically into its rest position, with the empty reservoir still assembled on the air expeller. However, the piston 21 remains in its dispensing position as a result of friction with the air chamber 22 and of the suction created in the reservoir 30, such that the cylindrical surface 614 slides over the inner lip 215 of the piston until said inner lip co-operates once again with the fluting 615. At this moment, the air chamber 22 is once again in communication with the surrounding air, and suction is no longer created by the return into the rest position. The piston 21 is thus also entrained towards its rest position. This makes it possible to close the reservoir after use.

Optionally, the unit formed by the upper body/dispenser head 1 and the empty reservoir 10 could be removed from the air expeller and replaced by a new unit that includes a full reservoir.

Appropriate applicator devices that may be used include those available from Aptar Pharma, France (UDS Monopowder). See for example international patent applications WO 2022/208014 and WO 2021/005311. Other examples of applicator devices that may be used in conjunction with compositions of the invention (especially those in the form of powders) include those described in US patent application US 2011/0045088, U.S. Pat. No. 7,722,566 (see e.g. FIGS. 1 and 7) and U.S. Pat. No. 5,702,362 and international patent application WO 2014/004400, the relevant disclosures of which documents are hereby incorporated by reference.

According to a further aspect of the invention, there is provided a process for the manufacturing of an applicator device comprising a composition of the invention, wherein said process comprises the step of loading said composition into a reservoir that is within, or is adjunct to, said applicator device.

According to a further aspect of the invention, there is provided a needle-free applicator that is suitable for administering a solid, amorphous mono-particulate powder composition of the invention into a body cavity of a human patient, which cavity includes a mucosal surface, wherein the applicator comprises:
(i) an (optionally opaque) reservoir that is within, or is adjunct, to said applicator comprising a composition of the invention;
(ii) an optional actuating means for generating a force upon actuation of the device by a user; and
(iii) a dispensing means through which, following said actuation, said powder composition may be dispensed.

According to another aspect of the invention, there is provided an applicator and/or dispenser device comprising one or more compositions of the invention in the form of a powder, which applicator or device may be actuated one or more times to deliver one or more compositions of the invention, each comprising an appropriate dose of active ingredient, upon each such actuation, which applicator/dispenser device comprises:
an outlet through which at least one composition is dispensed;
a means of externally generating a force (e.g. an air-flow) upon actuation of the device by a user;
at least one (optionally replaceable) reservoir that contains said one or more compositions of the invention, which reservoir is, or is capable of being placed, in direct or indirect communication with the dispenser outlet;
a displaceable, optionally reversible, sealing means in the device and/or the reservoir for retaining the one or more compositions within the reservoir until a composition is dispensed;
a mechanical opening system that co-operates with said sealing means such that a single composition of the invention is expelled mechanically by the forcing means when the device is actuated; and
optionally, a mechanism for re-sealing the device and/or the reservoir to retain further compositions within the reservoir until a further composition is to be dispensed.

According to a still further aspect of the invention there is provided an applicator and/or dispenser device comprising a single dose of a composition of the invention, suitable for dispensing that composition, which applicator/dispenser device comprises: a dispenser outlet;
an air expeller for generating a flow of air while the device is being actuated, said air expeller including a piston that slides in an air chamber between a rest position and a dispensing position;
said piston slides in airtight manner within said air chamber;
at least one reservoir that contains a dose of a composition of the invention, said reservoir including an air inlet that is connected to said air expeller;
a composition outlet that is connected to said dispenser outlet;
said air inlet including a displaceable sealing means (e.g. a retainer member) for retaining the composition in the reservoir until the composition is dispensed;
said composition outlet being closed by a closure element that is fitted in the composition outlet of the reservoir;
said device further including a mechanical opening system that co-operates with said closure element so as to expel it mechanically from its closed position while the device is being actuated; and
said piston of said air expeller, when in its rest position, co-operating in non-airtight manner with said air chamber.

In the latter aspect of the invention, it is preferred that:
(i) the air chamber within which said piston slides in airtight manner is substantially cylindrical;
(ii) the closure element is force fitted in the composition outlet of the reservoir;
(iii) said air chamber is in communication with the atmosphere in the rest position; and/or
(iv) said piston includes an inner lip that is suitable for co-operating with a cylindrical surface, said cylindrical surface includes fluting that co-operates in non-airtight manner with said inner lip of the piston in its rest position.

Such a nasal applicator or dispensing device is capable of providing for an appropriate and reproducible powder spray pattern and/or plume geometry that enables efficient delivery of said powder to the nasal cavity (e.g. a nostril).

In compositions of the invention, mean particle sizes may be presented as weight-, number-, or volume-, based mean diameters. As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). The term ‘volume based mean diameter’ is similar in its meaning to weight based mean diameter, but will be understood by the skilled person to include that the average particle size is characterised and defined from a particle size distribution by volume, i.e. a distribution where the existing fraction (relative amount) in each size class is defined as the volume fraction, as measured by e.g. laser diffraction. As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. Other instruments that are well known in the field may be employed to measure particle size, such as equipment sold by e.g. Malvern Instruments, Ltd (Worcestershire, UK), Sympatec GmbH (Clausthal-Zellerfeld, Germany) and Shimadzu (Kyoto, Japan).

Although particle size is not (or rather may not be) critical when compositions of the invention are formulated for administration e.g. perorally, topically, to the oral, ocular or other mucosae, or by injection or infusion, powder compositions of the invention will typically have a volume-based mean diameter (VMD) within the range of about 0.2 μm, such as about 0.5 μm (e.g. about 1 μm) up to about 1,000 μm (e.g. up to about 500 μm, such as about 400 μm or about 500 μm), and the appropriate particle size range may be selected based on the dosage form in which it is intended to include such compositions.

However, the skilled person will understand that, to allow for effective intranasal administration, powders will typically have a volume-based mean diameter (VMD) within the range of about 5 μm up to about 300 μm (e.g. up to about 200 μm). Depending on the applicator device that is employed, the VMD may be in the range of about 10 μm to about 100 μm, such as about 20 μm to about 60 μm.

Preferred particle size distributions for intranasal drug delivery may also include those in which the D10 is above about 3 μm and below about 75 μm (e.g. up to about 50 μm), such as greater than about 10 μm, and the D90 is between about 80 μm and about 1,000 μm (e.g. about 500 μm), such as less than about 100 μm. The skilled person will understand that the parameter ‘D10’ (or ‘Dv(10)’) means the size (or diameter) in a particle size distribution below which 10% of the total volume of material in the sample is contained. Similarly, the ‘D90’ (or ‘Dv(90)’) means the size below which 90% of the material is contained.

The skilled person will understand that, to allow for effective pulmonary administration, powders will typically have a VMD within the range of about 0.2 μm up to about 10 μm.

By powders having particle size distributions and VMDs within the above ranges, we include the bulk VMD and/or the emitted VMD, that is the particle size distribution when initially loaded into the device and/or when it is expelled therefrom, respectively.

Particle sizes may be measured by standard equipment, such as a dry (or a wet) particle size measurement technique, including dry dispersion technologies available from manufacturers such as Sympatec and Malvern.

Preferred particle shapes include spherical or substantially spherical, by which we mean that the particles possess an aspect ratio smaller than about 20, more preferably less than about 10, such as less than about 4, and especially less than about 2, and/or may possess a variation in radii (measured from the centre of gravity to the particle surface) in at least about 90% of the particles that is no more than about 50% of the average value, such as no more than about 30% of that value, for example no more than about 20% of that value.

Nevertheless, particles may be any shape, including irregular shaped (e.g. ‘raisin’-shaped), needle-shaped, disc-shaped or cuboid-shaped, particles. For a non-spherical particle, the size may be indicated as the size of a corresponding spherical particle of e.g. the same weight, volume or surface area.

The spray angle of emitted (dispensed) powder composition of the invention from a nasal applicator and/or a dispenser device should preferably be less than about 90°.

Wherever the word ‘about’ is employed herein in the context of amounts, for example absolute amounts, such as doses, weights, volumes, sizes, diameters, aspect rations, angles, etc., or relative amounts (e.g. percentages) of individual constituents in a composition or a component of a composition (including concentrations and ratios), timeframes, and parameters such as temperatures, pressure, relative humidities, etc., it will be appreciated that such variables are approximate and as such may vary by ±10%, for example ±5% and preferably ±2% (e.g. ±1%) from the actual numbers specified herein. This is the case even if such numbers are presented as percentages in the first place (for example ‘about 10%’ may mean±10% about the number 10, which is anything between 9% and 11%).

Compositions of the invention have the advantage that they are capable of being prepared and thereafter stored over a wide range of temperatures and relative humidities without significant loss in biological activity. Thus, compositions of the invention may be subject to low temperatures (e.g. below freezing) without impacting the amount of active ingredient that is administered to a subject. Further, compositions of the invention may have the advantage that they are more physically and chemically stable at higher temperature than relevant prior art compositions.

Compositions of the invention further may also have the advantage that they provide for higher bioavailability of active ingredients compared to prior art compositions. The compositions of the invention may provide for this higher bioavailability alongside a more rapid absorption, which will likely lead to a more rapid onset of action than such prior art and/or commercially-available compositions, and thus meets a significant medical need.

The compositions, pharmaceutical formulations, uses and methods described herein may also have the advantage that, in the treatment of the conditions for which the relevant active ingredient is known for, they may be more convenient for the first responder, physician and/or patient than, be more efficacious than, be less toxic than, have a broader range of activity than, be more potent than, produce fewer side effects than, have a lower inter-patient variability, or that it/they may have other useful pharmacological properties over, similar formulations or methods (treatments) known in the prior art, whether for use in the treatment of the aforementioned conditions by transmucosal, such as intranasal, administration or otherwise.

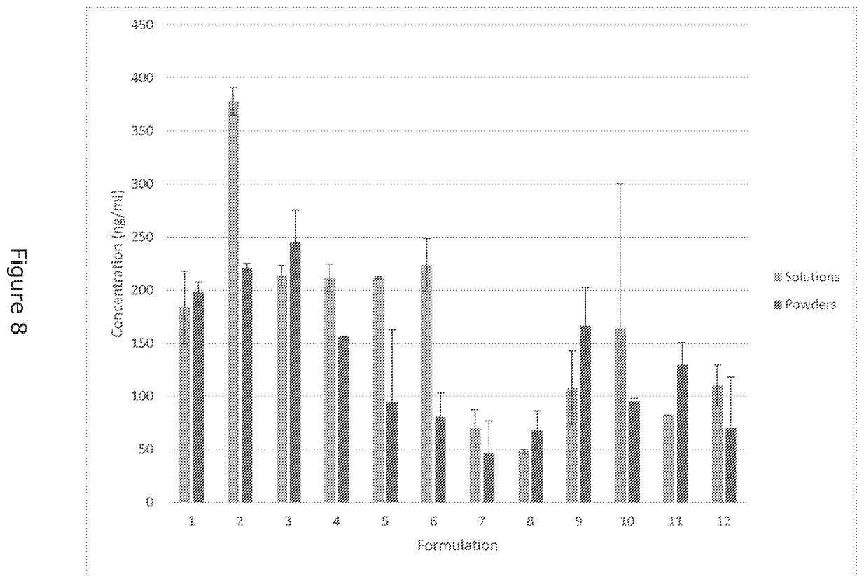

The invention is illustrated but in no way limited by way of the following example with reference to the figures in which FIGS. 1 to 7 represent drawings of actuator devices that may be used to dispense powder compositions, and FIGS. 8 and 9 show retained activity under different conditions of a SARS-CoV-2 RBD Spike protein following formation of a spray dried powder versus activity of the same protein to the initial solution prior to spray drying.

### EXAMPLE 1

### Compositions of the Invention I

Six formulations, each of approximately 2 g powder, were prepared by adding to 30 mL glass vials the required amounts of lyophilized β-galactosidase (lactase enzyme; 5000 units per mg; Merck/Sigma Aldrich Germany) and the following excipients: trehalose (Merck/Sigma Aldrich, Germany), maltodextrins (MD) IT12 and IT19 (Roquette, France), sucrose laurate D1216 (SL; Mitsubishi Chemicals, Japan) and HPMC K3 (Dupont, USA). The compositions were as shown (with individual components in wt %) in Table 1 below.

TABLE 1

| Formulation | Lactase | Trehalose | MD IT12 | MD IT19 | SL | HPMC |
|---|---|---|---|---|---|---|
| 1 | 0.61 | 40.0 | 52.3 | 0 | 3.05 | 0 |
| 2 | 1.22 | 40.0 | 51.7 | 0 | 3.05 | 0 |
| 3 | 1.22 | 40.0 | 0 | 53.3 | 1.49 | 0 |
| 4 | 0.61 | 40.0 | 47.4 | 0 | 3.05 | 4.80 |
| 5 | 0.61 | 40.0 | 52.3 | 0 | 3.05 | 0 |
| 6 | 0.61 | 40.0 | 0 | 53.9 | 1.49 | 0 |

The vials were then placed in a freezer kept at −20° C. Prior to shipping, the vials were placed in aluminum bags with a desiccant (molecular sieve) and placed in an insulated box with wet ice.

Spray-drying was performed at Xedev (Belgium) using a ProCepT spray-dryer with an extended column. From the six formulations prepared, a total of 12 samples were produced by altering the nozzle type and process conditions. An overview of the spray-drying process is shown in Table 2 below.

Prior to spray-drying, the six prepared formulations were dissolved in water with a resulting solid load (w/w %, water:solids) ranging between 5 and 10%. From these solutions, 0.5 mL was extracted from each vial into separate LC vials and placed in a freezer prior to shipping. These were the reference solutions used to determine the activity of the enzyme prior to spray-drying.

The feed-rate of the solutions was set to 4 g/min resulting in a process time between 1-5 min for the samples shown in Table 2. To reduce possible heat exposure during processing, the collection vessel was chilled using an ice bath during some of the runs.

TABLE 2

| | | | | | | | | Solid |
|---|---|---|---|---|---|---|---|---|
| | Formul'n | Nozzle | Loop | | Yield | Tin | Tout | L |
| ID | Used | Type | Type¹ | Chilled? | (%) | (° C.)² | (° C.)³ | (%) |
| 001 | 1 | Ultrasonic | Open | No | ~100 | 160 | 58 | 10 |
| 002 | 1 | Bi-fluid | Open | No | 68 | 120 | 47 | 10 |
| 003 | 5 | Ultrasonic | Open | No | 81 | 160 | 60 | 10 |
| 004 | 5 | Ultrasonic | Open | Yes | 82 | 160 | 59 | 10 |
| 005 | 3 | Ultrasonic | Open | No | 81 | 160 | 60 | 7.5 |
| 006 | 3 | Ultrasonic | Open | Yes | 83 | 160 | 58 | 7.5 |
| 007 | 2 | Ultrasonic | Closed | No | 81 | 160 | 65 | 7.5 |
| 008 | 2 | Ultrasonic | Closed | Yes | 85 | 160 | 66 | 7.5 |
| 009 | 4 | Ultrasonic | Closed | No | 76 | 160 | 66 | 10 |
| 010 | 4 | Ultrasonic | Closed | Yes | 75 | 160 | 68 | 10 |
| 011 | 6 | Bi-fluid | Closed | No | 81 | 120 | 50 | 5 |
| 012 | 6 | Bi-fluid | Closed | Yes | 81 | 120 | 50 | 5 |

### Notes

1. ‘Open’ means open to air; ‘Closed’ means under nitrogen gas
2. Inlet temperature, at the spray nozzle
3. Outlet temperature, at the exit of the cyclone

After spray-drying, the samples were placed in a freezer until shipping. The samples were shipped in the same conditions as described above, i.e. using desiccants and wet ice.

The enzyme activity assay was performed using 2-nitrophenyl β-D-galactopyranoside (ONPG; Merck/Sigma Aldrich, Germany) as a substrate for the enzyme.

Before analysis, 100 mg of each spray-dried material was transferred into separate LC vials and diluted to the same concentration as the reference solutions (see above).

The enzyme solutions, reference and spray-dried powders were diluted with PBS (pH 7.4) and thereafter 5 mM ONPG was added in excess. The UV/vis absorbance of the mixture was measured at 420 nm at 0.5, 1, 2, 3, 4 and 5 minutes. This was performed in duplicate.

The absorbance of the spray-dried solutions was compared to the corresponding reference solution using linear regression. The reference solution was designated as having 1000/activity while the quotient of the slope was used to determine the retained activity of the spray-dried solutions. The results are presented in Table 3 below. (Note—the results for Samples 001 and 002 were excluded as there are some uncertainties regarding the integrity of the reference sample.)

TABLE 3

| | Sample | | | | |
|---|---|---|---|---|---|
| | 003 | 004 | 005 | 006 | 007 |
| Time (mins) | Absorbance at 420 nm | | | | |
| 0.5 | 0.0585 | 0.0620 | 0.1280 | 0.1220 | 0.1165 |
| 1 | 0.1160 | 0.1175 | 0.2455 | 0.2335 | 0.2160 |
| 2 | 0.2260 | 0.2270 | 0.4740 | 0.4540 | 0.4125 |
| 3 | 0.3355 | 0.3370 | 0.7030 | 0.6685 | 0.6110 |
| 4 | 0.4445 | 0.4480 | 0.9245 | 0.8810 | 0.8050 |
| 5 | 0.5540 | 0.5580 | 1.1345 | 1.0850 | 0.9915 |
| Slope | 0.111 | 0.112 | 0.231 | 0.220 | 0.201 |
| Slope Ref. | 0.128 | 0.128 | 0.252 | 0.252 | 0.239 |
| Activity¹ | 87% | 87% | 92% | 88% | 84% |
| | Sample | | | | |
| | 008 | 009 | 010 | 011 | 012 |
| Time (mins) | Absorbance at 420 nm | | | | |
| 0.5 | 0.1165 | 0.0650 | 0.0695 | 0.0680 | 0.0625 |
| 1 | 0.2195 | 0.1230 | 0.1320 | 0.1360 | 0.1210 |
| 2 | 0.4240 | 0.2365 | 0.2530 | 0.2620 | 0.2380 |
| 3 | 0.6310 | 0.3490 | 0.3745 | 0.3890 | 0.3550 |
| 4 | 0.8320 | 0.4625 | 0.4965 | 0.5175 | 0.4730 |
| 5 | 1.0305 | 0.5750 | 0.6160 | 0.6425 | 0.5880 |
| Slope | 0.208 | 0.116 | 0.124 | 0.129 | 0.118 |
| Slope Ref. | 0.239 | 0.148 | 0.148 | 0.133 | 0.133 |
| Activity¹ | 87% | 78% | 84% | 97% | 88% |

Note
¹Activity = Slope Spry-Dried Sample/Slop of Reference Sample × 100

The results show that the loss in activity of the enzyme following a spray-drying process according to the invention is minimal.

### EXAMPLE 2

### Compositions of the Invention II

Twelve formulations, each comprising approximately 2.5-2.6 g powder, were prepared by adding the required amounts of lyophilized SARS-CoV-2 RBD Spike protein (L452R), His Tag (‘S protein’; SPD-C52He, ACRO Biosystems, US) and the following excipients: trehalose dihydrate (Pfanstiel, US), maltodextrin (MD) IT19 (Roquette, France), sucrose laurate D1216 (SL; Mitsubishi Chemicals, Japan), phosphate buffered saline (PBS) and pure deionized water (5% solid load), to a 100 mL glass flask. The formulations had the compositions a shown in Table 4 below (with individual components in wt %).

TABLE 4

| Formulation | S protein | Trehalose | MD IT19 | SL | PBS |
|---|---|---|---|---|---|
| 7 | 0.0009 | 20.0 | 73.0 | 3.0 | — |
| 8 | 0.0009 | 40.0 | 53.0 | 3.0 | — |
| 9 | 0.0009 | 60.0 | 33.0 | 3.0 | — |
| 10 | 0.0009 | 80.0 | 13.0 | 3.0 | — |
| 11 | 0.0009 | 20.0 | 76.0 | — | — |
| 12 | 0.0009 | 80.0 | 16.0 | — | — |
| 13 | 0.0009 | 20.0 | 57.0 | — | 19 |
| 14 | 0.0009 | 61.0 | 16 | — | 19 |
| 15 | 0.0009 | 20.0 | 76.0 | 3.0 | — |
| 16 | 0.0009 | 40.0 | 53.0 | 3.0 | — |
| 17 | 0.0009 | 60.0 | 33.0 | 3.0 | — |
| 18 | 0.0009 | 80.0 | 53.0 | 3.0 | — |

4% nominal water content in final powder.

Spray-drying was performed using a ProCepT spray-dryer with an extended column using both an ultrasonic and a bi-fluid nozzle. An overview of the spray-drying process is shown in Table 5 below.

Prior to spray-drying, 0.8 mL of the solutions were extracted from each vial into separate LC vials and placed in a freezer (−20° C.). These were the reference solutions used to determine the initial activity of the protein prior to spray-drying.

The feed-rate of the solutions was set to 4 g/min resulting in a process time around 13 min for the samples shown in Table 5.

TABLE 5

| Formulation | Nozzle Type | Recovery(%) | Tᵢₙ (° C.) | Solid L (%) |
|---|---|---|---|---|
| 7 | Ultrasonic | 68 | 160 | 5 |
| 8 | Ultrasonic | 87 | 160 | 5 |
| 9 | Ultrasonic | 82 | 160 | 5 |
| 10 | Ultrasonic | 79 | 160 | 5 |
| 11 | Ultrasonic | 77 | 160 | 5 |
| 12 | Ultrasonic | 88 | 160 | 5 |
| 13 | Ultrasonic | 101 | 160 | 5 |
| 14 | Ultrasonic | 88 | 160 | 5 |
| 15 | Bi-fluid | 72 | 120 | 5 |
| 16 | Bi-fluid | 74 | 120 | 5 |
| 17 | Bi-fluid | 71 | 120 | 5 |
| 18 | Bi-fluid | 71 | 120 | 5 |

After spray-drying, the samples were placed in sealed aluminum pouches with desiccant and stored at −20° C. until analysis. Additional powder was packed in the same way and stored for 4 weeks at four different temperatures, −20° C., −5° C., 20° C. and 40° C.

The protein activity was analyzed using a SARS-CoV-2 Spike Protein Titer Assay Kit, ELISA (RAS-A020-96TEST, ACRO Biosystems, US). Before analysis, spray-dried material and the initial solutions were diluted to a target concentration around 0.2 ng/mL and all samples were analyzed in duplicates.

The measured retained activity after spray drying, powder in relation to initial solution, was in the range of 30% (acceptable loss) to 100% (minimal loss), as shown in FIG. 8.

A second analysis was performed after 4 weeks of storage. Noting some variability both in between, and within, the analysis (including higher activity upon storage and variability between duplicates), it was evident that activity was retained in all samples stored at all temperatures, as shown in FIG. 9.

1. A pharmaceutically-acceptable composition, which composition is in the form of a solid, amorphous mono-particulate powder composition comprising a mixture of:
(a) a pharmacologically-effective dosage amount of at least one biopharmaceutical drug compound; and
(b) a pharmaceutically-acceptable carrier material, which carrier material comprises a combination of a disaccharide and a polymeric material.

2. A composition as claimed in claim 1, wherein the polymeric material comprises a maltodextrin.

3. A composition as claimed in claim 1 or claim 2, wherein the disaccharide is selected from the group consisting of maltitol, trehalose, sucralose, sucrose, isomalt, maltose and lactose.

4. A composition as claimed in claim 3, wherein the disaccharide comprises a lactose or trehalose.

5. A composition as claimed in any one of the preceding claims, wherein the carrier material comprises a combination of trehalose and maltodextrin 19DE.

6. A composition as claimed in any one of the preceding claims, wherein the ratio of disaccharide:polymer by weight, based on the total weight of the composition, is in the range of about 10:1 and about 1:10.

7. A composition as claimed in claim 6, wherein the ratio of disaccharide:polymer is in the range of about 2:1 to about 1:8.

8. A composition as claimed in any one of the preceding claims, wherein the lowest measurable glass transition temperature of the composition is at least about 10° C. when measured at a relative humidity of up to about 35%.

9. A composition as claimed in any one of the preceding claims, wherein the composition further comprises a sucrose ester.

10. A composition as claimed in claim 9, wherein the sucrose ester comprises sucrose monolaurate.

11. A composition as claimed in any one of the preceding claims wherein the pharmacologically-effective dosage amount of the at least one biopharmaceutical drug compound is no more than about 100 mg.

12. A composition as claimed in any one of the preceding claims wherein at least one biopharmaceutical drug compound is selected from the group: a protein, as oligopeptide, a polypeptide, an enzyme, an antibody, a vaccine and a nucleotide.

13. A composition as claimed in any one of the preceding claims which is suitable and/or adapted for nasal delivery.

14. A composition as claimed in claim 13, wherein the particle size distribution of the powder includes a D10 that is above about 3 μm.

15. A composition as claimed in claim 13 or claim 14, wherein the powder has a particle size distribution that includes a volume-based mean diameter within the range of about 10 μm and about 100 μm.

16. A composition as claimed in any one of claims 13 to 15, wherein the biopharmaceutical drug compound is a vaccine.

17. A composition as claimed in any one of claims 1 to 12 which is suitable and/or adapted for peroral delivery.

18. A composition as claimed in claim 17, wherein the biopharmaceutical drug compound is an enzyme.

19. A composition as claimed in any one of claims 1 to 12 which is dissolved in a pharmaceutically-acceptable solvent for delivery by injection or by infusion.

20. A composition as claimed in claim 19, wherein the biopharmaceutical drug compound is an antibody.

21. A process for the manufacturing of a composition as defined in any one of the preceding claims, wherein said process comprises the steps of:
(i) mixing together the one or more biopharmaceutical drug compounds and pharmaceutically-acceptable carrier materials, in an appropriate volatile solvent,
(ii) spray-drying the mixture from step i).

22. A composition obtainable by a process as defined in claim 21.

23. A nasal applicator device suitable and/or adapted for delivery of a composition as defined in any one of claims 1 to 16 or 22 to the nose, which comprises, or is adjunct and/or attached to, a reservoir, within which reservoir said composition is contained.

24. A process for the manufacturing of an applicator device as claimed in claim 23, which comprises a process as claimed in claim 21 followed by loading the composition so formed into a reservoir within, or adjunct or attached to, said applicator device.

25. A composition as defined in any one of claims 1 to 20 or 22 for use in the treatment of a condition for which the at least one biopharmaceutical drug compound that is/are included therein is/are useful for.

26. The use of a composition as defined in any one of claims 1 to 20 or 22 for the manufacture of a medicament for the treatment of a condition for which the at least one biopharmaceutical drug compound that is/are included therein is/are useful for.

27. A method of treatment of a condition for which the at least one biopharmaceutical drug compound that is/are included within composition as defined in any one of claims 1 to 20 or 22 is/are useful for, which method comprises the administration of a composition of the invention to patient suffering from, or susceptible to, said condition.
